(19) United States
(12) Patent Application Publication
(10) Pub. No.: US 2016/0161459 A1
(43) Pub. Date: Jun. 9, 2016

(54) APPARATUS FOR DETECTION AND DELIVERY OF VOLATILIZED COMPOUNDS AND RELATED METHODS

(71) Applicants: R. ROUSE, San Diego, CA (US); CDX, INC., La Jolla, CA (US)

(72) Inventor: Richard James Douglas Rouse, San Diego, CA (US)

(21) Appl. No.: 14/905,780

(22) PCT Filed: Jul. 14, 2014
(86) PCT No.: PCT/US14/46500

§ 371 Date: Jan. 15, 2016

Related U.S. Application Data
(60) Provisional application No. 61/846,996, filed on Jul. 16, 2013.
(60) Provisional application No. 61/864,515, filed on Aug. 9, 2013.
(60) Provisional application No. 61/864,517, filed on Aug. 9, 2013.
(60) Provisional application No. 61/927,992, filed on Jan. 16, 2014.

(51) Int. Cl. G01N 33/00; G01N 1/28

(52) U.S. Cl. CPC G01N 33/0098; G01N 1/28

(57) ABSTRACT

The present invention relates generally to devices and related methods for the volatilization, separation, mobilization and detection of volatile components of a sample. The present invention also relates to devices and methods for the fractionation of volatile sub-components. The volatile component subfractions can be selectively delivered to a subject.

### CROSS REFERENCE TO RELATED APPLICATIONS

This application claims the benefit of priority to U.S. Provisional Patent Application Ser. No. 61/846,996, filed Jul. 16, 2013 and entitled “A Portable Chemical Analysis Method by Volatilization and Sublimation”; to U.S. Provisional Patent Application Ser. No. 61/864,515, filed Aug. 9, 2013 and entitled “A Portable Gas Chromatographic Analysis and Distribution System”; to U.S. Provisional Patent Application Ser. No. 61/864,517, filed Aug. 9, 2013 and entitled “A Portable Gas Chromatographic Analysis and Distribution System for the Extraction and Delivery of Compounds in Cannabis”; and to U.S. Provisional Patent Application Ser. No. 61/927,992, filed Jan. 16, 2014 and entitled “A Method for Therapeutic Development, Preparation and Administration”; all of which are incorporated by reference.

### FIELD OF THE INVENTION

The present invention is in the technical field of devices and related methods for the volatilization, separation, mobilization and detection of volatile components of a sample. The present invention also relates to devices and methods for the fractionation of volatile components.

### BACKGROUND OF THE INVENTION

The electronic nose was developed in order to mimic human olfaction for detecting odors and typically consists of head space sampling by a sensor array. The response pattern of the sensor array toward to a sample is compared against a response pattern for a known compound or compounds to identify the sample.

Volatility is the tendency of a substance to vaporize or sublimate. Most compounds are not volatile enough at ambient conditions to be detected by electronic nose sensors. Consequently, there is a need for devices and methods that enhance the volatiles in a sample to improve detection by electronic nose sensors. Moreover, there is a need to fractionate the volatile compounds for detection, analysis and delivery.

Personalized medicine is a growing field. Appropriate dosing of a pharmaceutical agent to a subject is a challenge. In addition, there are many chemical agents, supplements and the like that are consumed for their health effects, but the consumer often has little control over identifying and administering the active components of these compounds. Moreover, there are no integrated resources for both the detection and administration of such compounds.

Cannabinoids have been found to have antioxidant and neuroprotectant properties (see U.S. Pat. No. 6,630,507), which make them useful in the treatment of a wide range of ailments. Studies have shown that cannabis can help patients inhibit cell growth in tumor/cancer cells, with Alzheimer's Disease, reduce blood sugar levels, reduce vomiting and nausea, reduce seizures and convulsion, reduce inflammation, reduce risk of artery blockages, prevent migraines, with tourette's/OCD, with glaucoma, treat fungal infection, kill or slows bacterial growth, relieve pain, relieve anxiety, aid sleep, suppress muscle spasms, stimulate appetite (or suppress it with certain cannabinoinds), promote bone growth, protect nervous system degeneration and fight speech impediments (stutters).

Gas Chromatography/Mass Spectroscopy has been used to analyze cannabinoids (U.S. Pat. No. 5,252,490). Gas Chromatography/Flame Ionization Detection (GC-FID) as well as High Performance Liquid Chromatography (HPLC) have also been used. U.S. Pat. No. 6,403,126 describes the use of a chromatographic column with a number of solvents to prepare a cannabis extract. US Patent Pub. No. 20070077660, uses extraction solvent and thin layer chromatography along with staining methods to detect a cannabinoid in cannabis. These methods are laboratory based methods that are highly specialized, typically require large capital intensive equipment, relatively expensive, and not easily portable.

There are 483 identifiable chemical constituents known to exist in the cannabis plant, and at least 85 different cannabinoids have been isolated from the plant. Studies have shown that by controlling the amount of specific active consumed, one can minimize side effects and better achieve the desired physiological effects from the desired chemicals in a cannabis sample. No device that can enable this type of chemical detection and delivery control is available today.

There also exists a need for alternative methods to analyze the quality and composition of a cannabis sample. Moreover, there exists a need for methods to extract one or more components of a cannabis sample and help a patient achieve the desired cannabinoid therapeutic profile and dosage.

### SUMMARY OF THE INVENTION

Provided herein are devices for analyte detection including a sample preparation module, the sample preparation module including a volatilization element, in fluid communication with a detection module, the detection module including a sensor array. The sensor array can detect a plurality of analytes. The volatilization element can include one or more and in any combination of a heating apparatus, a sonication apparatus, a stirring apparatus, a grinding apparatus or a mechanical shearing apparatus. The sensor array can be a conducting polymer sensor array, a carbon nanotube sensor array, a MEMS cantilever sensor array, a surface acoustic wave sensor array, a metal-oxide sensor array, an electrochemical sensor array, a quartz crystal microbalance sensor array, a colorimetric sensor array, a fluorescence sensor array, an infrared sensor array, a MOSFET sensor array or a catalytic bead sensor array. The devices can further include a mobilization module that directs flow of the volatile component from the sample preparation module to the detection module. The mobilization module can be disposed in any orientation between modules. The mobilization module can include a pump and the pump can be a displacement pump, a peristaltic pump, a diaphragm pump or a vacuum driven pump. The devices can include one or more filters, which can be disposed in any orientation between the modules. The filter material can include carbon, polyester, polycarbonate, cellulose nitrate, mixed cellulose ester, nylon, polytetrafluoroethylene, polypropylene, aluminum oxide, polyether sulfone, or nitrocellulose.

Provided herein are any of the above disclosed devices further including a distribution module, the distribution module including one or more control valves and one or more connection ports. At least one control valve can be controlled by information received from the sensor array. The one or more control valves can be a rotary valve, a solenoid valve, a pinch valve, or a hydrogel valve. At least one of the control valves can be computer controlled. At least one of the one or more connection ports can be controlled mechanically. The device can further include one or more filters, as disclosed above. The one or more connection ports can be connected to one or more components including a detection module, a dispensing component module or a sensor device. The device can further include a mobilization module as disclosed above.

Provided herein are also methods of analyte detection that include (a) volatilizing a sample resulting in a volatile component, (b) separating the volatile component from the remaining sample, and (c) contacting the volatile component with a sensor array, wherein the sensor array detects the analyte in the volatile component when the analyte is present in sufficient concentration. The volatilizing of the sample can be performed by one or more of heating, sonicating, stirring, grinding or mechanical shearing. The volatilizing of the sample, such as by heating, can be modulated to selectively volatilize at least one analyte. The methods can further include a filtration step between volatilizing the sample and detecting the analyte in the volatile component. The sample can be an environmental sample, such as water, air, soil or vegetation. The method sample can be a biological specimen such as exhaled breath, sputum, blood or component thereof, urine, feces, or tissue. The sample can be a pharmaceutical preparation and the analyte can be a pharmaceutically active component. The sample can be Cannabis. The analyte can be a cannabinoid, a terpenoid, or a flavonoid. The cannabinoid can be Tetrahydrocannabivarin (THCV), Delta-8-tetrahydrocannabinol (delta-8-THC), Delta-9-tetrahydrocannabinol (THC), Cannabichromene (CBC), Cannabidiol (CBD), Cannabigerol (CBG), or Cannabinol (CBN). The terpenoid can be Linalool, d-Limonene, Myrcene, alpha-Pinene, and Beta-Caryophyllene. The flavanoid can be Beta-sitosterol, Apigenin, Cannflavin A, and Quercetin.

Also provided are methods for regulating delivery of a volatile component including (a) volatilizing a sample resulting in a volatile component, (b) separating the volatile component from the remaining sample, (c) contacting the volatile component with a sensor array, wherein the sensor array detects an analyte in the volatile component when the analyte is present in sufficient concentration, and (d) regulating delivery of the volatile component based on a preset level of analyte delivery. The sensor array can detect a plurality of analytes. The volatilizing of the sample can be by one or more of heating, sonicating, stirring, grinding or mechanical shearing. The volatilizing of the sample, such as by heating, can be modulated to selectively volatilize at least one analyte. The sample can be a pharmaceutical preparation and the analyte can be a pharmaceutically active component. The sample can be Cannabis. The analyte can be a cannabinoid, a terpenoid, or a flavonoid. The cannabinoid can be Tetrahydrocannabivarin (THCV), Delta-8-tetrahydrocannabinol (delta-8-THC), Delta-9-tetrahydrocannabinol (THC), Cannabichromene (CBC), Cannabidiol (CBD), Cannabigerol (CBG), or Cannabinol (CBN). The terpenoid can be Linalool, d-Limonene, Myrcene, alpha-Pinene, and Beta-Caryophyllene. The flavanoid can be Beta-sitosterol, Apigenin, Cannflavin A, and Quercetin. The methods can further include a filtration step (a) between volatilizing the sample and contacting the volatile component with a sensor array or (b) between the sensor array and the delivery of the volatile component. The volatile component can be collected and, optionally, concentrated before delivery. Concentration can be achieved by condensation of the gas or vapor. The delivery can be to a subject, which can be at a preset level. The preset level of analyte delivery can be adjustable by the subject. The subject can be provided sample-related information. The sample-related information can be one or more of sensor information, clinical history information, method of action information, reported effects information, administration information, sample composition information, therapeutic preparation information, or marketplace information.

Also provided are methods for regulating delivery of a subfraction of a volatile component including (a) volatilizing a sample resulting in a volatile component, (b) separating the volatile component from the remaining sample, (c) contacting the volatile component with a sensor array, wherein the sensor array detects an analyte in the volatile component when the analyte is present in sufficient concentration, (d) fractionating the volatile component into a plurality of subfractions, and (e) regulating delivery of at least one subfraction of the volatile component based on a preset level of analyte delivery. The sensor array can detect a plurality of analytes. The subfraction can include an increased concentration of at least one analyte. Fractionating the volatile component into subfractions can be performed by modulated volatilization, such as by modulated heating, to selectively volatilize at least one analyte. The volatilizing of the sample can be performed by one or more of heating, sonicating, stirring, grinding or mechanical shearing. The sample can be a pharmaceutical preparation and the analyte can be a pharmaceutically active component. The sample can be Cannabis. The analyte can be a cannabinoid, a terpenoid, or a flavonoid. The cannabinoid can be Tetrahydrocannabivarin (THCV), Delta-8-tetrahydrocannabinol (delta-8-THC), Delta-9-tetrahydrocannabinol (THC), Cannabichromene (CBC), Cannabidiol (CBD), Cannabigerol (CBG), or Cannabinol (CBN). The terpenoid can be Linalool, d-Limonene, Myrcene, alpha-Pinene, and Beta-Caryophyllene. The flavanoid can be Beta-sitosterol, Apigenin, Cannflavin A, and Quercetin. The methods can further include a filtration step (a) between volatilizing the sample and contacting the volatile component with a sensor array or (b) between the sensor array and the delivery of the volatile component. The volatile component can be collected and optionally, concentrated before delivery. Concentration can be achieved by condensation of the gas or vapor. The delivery can be to a subject, which can be at a preset level. The preset level of analyte delivery can be adjustable by the subject. The subject can be provided sample-related information. The sample-related information can be one or more of sensor information, clinical history information, method of action information, reported effects information, administration information, sample composition information, therapeutic preparation information, and marketplace information.

As used herein, “volatilization element” means any apparatus and its associated controller that can increase the volatile component of the sample. “Volatilizing” means a process that enhances the release of a volatile component.

As used herein, “in fluid communication” means that two or more modules are configured to allow passage of the volatile component from one module to the other.

As used herein, an “analyte” is any molecule that can be volatilized and detected by a sensor array.

As used herein, “concentrated” means that the volatile component is made less dilute. For example, a vapor can be concentrated by condensation. As used herein, “condensation” means the conversion of a gas or vapor to a liquid.

As used herein, “Cannabis” means any preparation of the cannabis plant that includes psychoactive constituents. Pharmacologically, the principal psychoactive constituent of cannabis is tetrahydrocannabinol (THC); it is one of 483 known compounds in the plant, including at least 84 other cannabinoids, such as cannabidiol (CBD), cannabinol (CBN), tetrahydrocannabivarin (THCV), and cannabigerol. As used herein, “a sample of Cannabis” means any sample that includes Cannabis, including any preparation of the plant such as food items and lotions.

### BRIEF DESCRIPTION OF THE DRAWINGS

The present invention is illustrated and described herein with reference to the various drawings, in which like reference numbers are used to denote like system components/method steps, as appropriate, and in which:

FIG. 1 shows a system overview of a volatilization and detection device for analyte detection in the volatile component of a sample using an electronic nose;

FIG. 2 shows a perspective exploded view of an embodiment of a sample preparation module and solid perspective views of the same assembled;

FIG. 3 shows a perspective view of a sample preparation module and its engagement with a connector to a detection module;

FIG. 4 shows a bottom view of a sensor chamber;

FIG. 5 shows an exploded view of a sensor chamber with gasket and sensor array chip;

FIG. 6 shows a bottom view of a sensor array chip;

FIG. 7 shows a pogo pin array for a sensor array chip;

FIG. 8 shows a cartoon demonstrating, in an embodiment, sensor function;

FIG. 9 shows the distinct resistance change for an array of sensors for analytes X, Y, and Z;

FIG. 10 shows a representative diagram of a volatilization and detection device;

FIG. 11 shows a system overview of a volatilization and detection device further including a distribution module and a dispensing component module;

FIG. 12 shows a diagram illustrating control electronics for devices of the invention;

FIG. 13 shows a diagram illustrating control electronics for devices of the invention;

FIG. 14 shows a perspective view of an embodiment of the inventive device;

FIG. 15 shows a perspective view of another embodiment of the inventive device;

FIG. 16 shows a diagram of sample-related information populating a database and accessed by an I/O device;

FIG. 17 shows a representative work flow for using the inventive device for the administration of a therapeutic;

FIG. 18 shows a representative work flow for using the inventive device for the administration of cannabis;

FIG. 19 shows the response of a sensor to repeated exposure of a volatile component;

FIG. 20 shows volatile component detection profiles for samples A, B and C;

FIG. 21 shows principal component analysis of volatile component detection profiles for samples A, B and C;

FIG. 22 shows volatile component detection profiles for samples D through L;

FIG. 23 shows a linear concentration response for detection of a THC Simulant;

FIG. 24 shows a linear concentration response for detection of a CBD Simulant;

FIG. 25 shows an example smartphone screenshot for a user interface;

FIG. 26 shows an example smartphone screenshot for a user interface;

FIG. 27 shows an example smartphone screenshot for a user interface;

FIG. 28 shows an example smartphone screenshot for a user interface;

FIG. 29 shows an example smartphone screenshot for a user interface;

FIG. 30 shows an example smartphone screenshot for a user interface;

FIG. 31 shows an example smartphone screenshot for a user interface;

FIG. 32 shows an example smartphone screenshot for a user interface;

FIG. 33 shows an example smartphone screenshot for a user interface;

FIG. 34 shows an example smartphone screenshot for a user interface;

FIG. 35 shows an example smartphone screenshot for a user interface;

FIG. 36 shows an example smartphone screenshot for a user interface;

FIG. 37 shows an example smartphone screenshot for a user interface;

FIG. 38 shows an example smartphone screenshot for a user interface; and,

FIG. 39 shows an example smartphone screenshot for a user interface.

### DETAILED DESCRIPTION OF THE INVENTION

In various exemplary embodiments, the present invention provides generally devices for the detection, separation and delivery of volatile components and subfractions thereof and related methods. The invention devices and methods enhance the release of volatile components of a sample for improved detection using electronic nose technology. Furthermore, the invention provides devices and methods for the fractionation of volatile compounds for improved sensor detection. In addition, the invention provides devices and methods for the fractionation of volatile components for selective delivery of analytes, such as to a receptacle or to a subject.

In suitable embodiments, the devices of the invention include a sample preparation module that includes a volatilization element, the sample preparation module in fluid communication with a detection module. The sample preparation module includes a chamber suitably configured to contain a sample when introduced to the sample preparation module.

In an embodiment the volatilization element is a heating apparatus. In another embodiment, the volatilization element is a sonication apparatus. In another embodiment, the volatilization element is a grinding apparatus. A plurality of volatilization elements can be employed. Other volatilization elements are known to those skilled in the art that impart energy to a sample such as those that introduce mechanical shear. Volatilization elements can include stirrers, fans, macerators, etc. For example, heating increases the amount of detectable analyte available by increasing the release of a volatile component of a sample. Programmable heating at different temperatures allows for differential release of volatile compounds from a sample either as a function of temperature or time, thereby potentially decreasing the presence of volatile compounds competing for detection of an analyte of interest.

The heating apparatus can include a heating element and a heating controller. Heating can be accomplished by, for example, thermal conduction, convection, or thermal radiation. In conduction heating, the sample is placed on a heat conducting surface such as a metal plate that is then heated to release the volatile components. In convection heating, the sample does not contact the heating element. Instead, hot gas, such as air, passes through the sample to cause release of the volatile components. This heating method can release more volatile components than conduction heating. In radiation heating, the sample is exposed to radiant energy to cause release of the volatile components. The energy can be provided by a superheated thermal mass placed around it, or from a visible bright light source like the sun or a laser. Thermal radiation heating permits uniform heating of a sample without diluting the volatile component produced.

The sample can be any liquid, solid or gas. The volatile component released from the sample can be a gas, a vapor, an aerosol or a suspension. An aerosol is a colloid of fine solid particles or liquid droplets, in air or another gas. A suspension is a heterogeneous mixture containing solid particles that are sufficiently large for sedimentation.

The sample can be an environmental sample or a biological specimen. Examples of environmental specimens include, but are not limited to air, water, soil, and vegetation. Examples of biological specimens include, but are not limited to, exhaled breath, sputum, blood and its components (e.g. serum, plasma, white blood cells, red blood cells, etc.), urine, feces, and tissues. The sample can be a chemical or pharmaceutical product.

The sample can be Cannabis or any preparation including Cannabis. Pharmacologically, the principal psychoactive constituent of cannabis is tetrahydrocannabinol (THC); it is one of 483 known compounds in the plant, including at least 84 other cannabinoids, such as cannabidiol (CBD), cannabinol (CBN), tetrahydrocannabivarin (THCV), and cannabigerol.

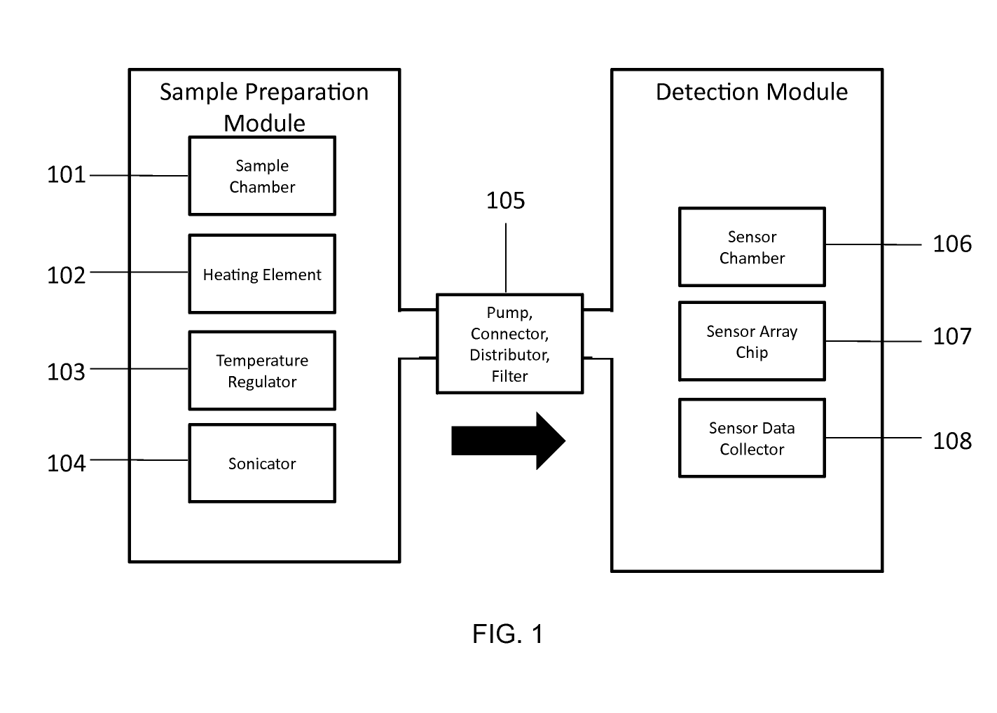

A general system overview of an embodiment of the device of the invention is outlined in FIG. 1. The device includes a sample preparation module and a detection module. In the embodiment shown, the sample preparation module includes a sample chamber 101, and a volatilization element such as a heating apparatus, which includes a heating element 102 and temperature regulator 103. In a suitable embodiment the preparation module can have a sonication apparatus 104. In a suitable embodiment, the sample preparation module can have both a heating apparatus and a sonication apparatus. In a suitable embodiment, the sample preparation module can have both a heating apparatus and a grinding apparatus.

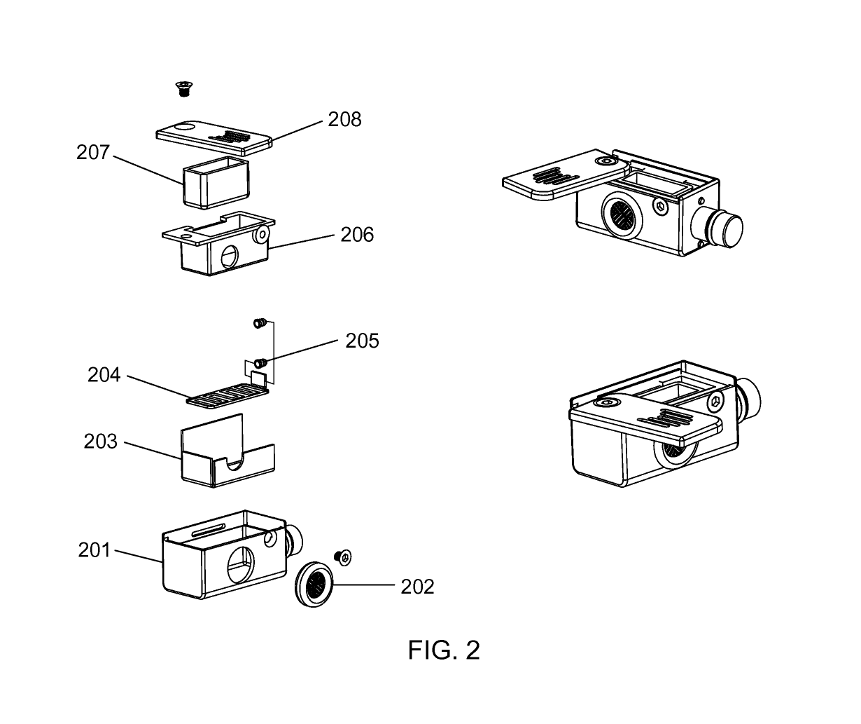

An example of a sample preparation module is shown in FIG. 2 in an exploded view and two perspective views of the assembled module. In this embodiment, the sample preparation module includes a main housing 201 with a filter 202, an insulating compartment 203, a flexible heating element 204, pogo pins 205 wired to the flexible heating element 204, a pan 206 that is heated by the flexible heating element 204, a sample pod 207, which can be made of glass, and a sample cover 208 that can be hingeably connected to the main housing 201 so that the cover can be slid out of the way in order for the operator to insert sample and then close the sample preparation module. Other configurations are within the skill of the artisan. For example, the sample preparation module can have a sliding cover.

FIG. 3 shows another perspective view of the exemplary sample preparation module and its connections to the detection module. The sample preparation module can include connectors for a heating element 301, a temperature regulator 302, and a sonication element or grinding element 303. The heating element and the sonication (or grinding) element can be included in the sample preparation housing 304. Other volatilization elements and combinations thereof are possible. As shown in the figure, the sample preparation module can be disposed to swivel into the housing of the device, but the skilled artisan would recognize that there are numerous other configurations.

Returning to FIG. 1, a mobilization module 105 can be used to efficiently transfer the volatile component of the sample from the sample preparation module to the detection module, wherein the mobilization module is in fluid communication with the other modules. The mobilization module 105 can be a microfluidic network. While the mobilization module 105 is shown in the drawing between the sample preparation module and the detection module, those skilled in the art recognize that the mobilization module can also be disposed in-line before the sample preparation module or after the detection module as long as the direction of the flow of the volatile component is from the sample preparation module to the detection module. The mobilization module can include a pump.

The mobilization module 105 can also include a distributor for connecting and bypassing sample flow to other detection or sample processing modules. The detection module can also include a filter 106. The filter can comprise carbon, polyester, polycarbonate, cellulose nitrate, mixed cellulose ester, nylon, polytetrafluoroethylene (PTFE), polypropylene, aluminum oxide, polyether sulfone (PES), or nitrocellulose. In another embodiment, the filter can be a high-efficiency particulate air or HEPA air filter. The skilled artisan recognizes that other filter material is available and that filters can be suitably placed optionally in and between each module and component. One example is to have carbon material to function as a filter for a gas.

The detection module includes a sensor chamber or detection chamber 106. The detection chamber includes a sensor array. The sensor array can include a sensor array chip 107 and a sensor data collector 108.

In an embodiment, the sensor apparatus is an electronic nose. Electronic nose devices perform odor detection through the use of an array of cross-reactive sensors in conjunction with pattern recognition methods. In contrast to the “lock-and-key” model, each sensor in the electronic nose device is widely responsive to a variety of analytes. In this architecture, each analyte produces a distinct signature from the array of broadly cross-reactive sensors. This configuration allows to considerably widen the variety of compounds to which a given matrix is sensitive, to increase the degree of component identification and, in specific cases, to perform an analysis of individual components in complex multi-component mixtures. Pattern recognition algorithms can then be applied to the entire set of signals, obtained simultaneously from all the sensors in the array, in order to acquire information on the identity, properties and concentration of the vapor exposed to the sensor array. Examples of algorithms and computer controlled systems for olfactometry include, but are not limited to, those disclosed in U.S. Pat. Nos. 6,411,905, 6,606,566, 6,609,068, 6,620,109, 6,767,732, 6,820,012, and 6,839,636. The analyte can be organic or inorganic. Where the analyte is organic, that is a Volatile Organic Compound (VOC), various devices and methods for VOC detection and analysis are disclosed, for instance, in U.S. Pat. Nos. 6,173,602, 6,319,724, 6,411,905, 6,467,333, 6,606,566, 6,609,068, 6,620,109, 6,703,241, 6,767,732, 6,820,012, 6,839,636, and 6,841,391.

Other patents relating to the inventive device include U.S. Pat. Nos. 5,571,401, 5,698,089, 5,788,833, 5,891,398, 5,911,872, 5,951,846, 5,959,191, 6,010,616, 6,013,229, 6,093,308, 6,170,318, 6,290,911, 6,455,319, 6,653,489, 6,610,367, 6,631,333, 6,759,010, 6,773,926, 6,962,675, 7,061,061, 7,122,152, 7,144,553, and 7,189,353.

The more commonly used sensors for electronic noses include, but are not limited to, conducting polymer sensors, carbon nanotube sensors, MEMS cantilevers, surface acoustic wave sensors, metal-oxides, electrochemical sensors, quartz crystal microbalance (QCM), colorimetric sensors, fluorescence sensors, infrared sensors, MOSFET sensors and catalytic beads.

Still other examples of sensor arrays are described in U.S. Pat. No. 8,034,222 (Conducting Polymer Nanowire Sensors), U.S. patent application Ser. No. 12/514,050 (Metal Nanoparticles Decorated Carbon Nanotubes for Gas Sensors), U.S. patent application Ser. No. 13/113,623 (Synthesis of Nanopeapods by Galvanic Displacement of Segmented Nanowires), and U.S. patent application Ser. No. 13/111,452 (Metal and Metal Oxides Co-Functionalization SWNT's as High Performance Gas Sensors).

In a suitable embodiment of this invention, the detection module includes a sensor array described in International Publication Number WO99/08105 entitled “Techniques and Systems for Analyte Detection”. In this embodiment, unique thin films of carbon black-organic polymer composites are deposited across two metallic leads, and swelling-induced resistance changes of the films signals the presence of certain vapors. To identify and classify volatile components, arrays of such vapor sensing elements are constructed, with each element containing the same carbon black conducting phase but a different organic polymer as the insulating phase thereby absorbing different analytes with different affinities.

Other types of electronic noses include mass spectrometry, ultra-fast gas chromatography, or Field Asystematic Ion Mobility Spectrometer (FAIMS) technology.

An analyte is any molecule that can be volatilized and detected by a sensor array. For example, if the sample is air, water, vegetation or soil, the analyte can be an environmental contaminant. The environmental contaminant can be a pesticide, a toxin or a heavy metal. The analyte can be an allergen, which could exist in any sample type. Allergens include, but not limited to, cat dander, peanut protein, and pollen. The analyte can be a marker of ripeness for vegetation. The analyte can be a pathogen, such as bacteria, virus or fungus. The analyte can be a biological molecule such a protein, a nucleic acid, a lipid or a sugar. The biological molecule can be a marker for a disease or disorder, such as cancer.

Where the sample is Cannabis, analytes can include cannabanoids, terpenoids and flavonoids.

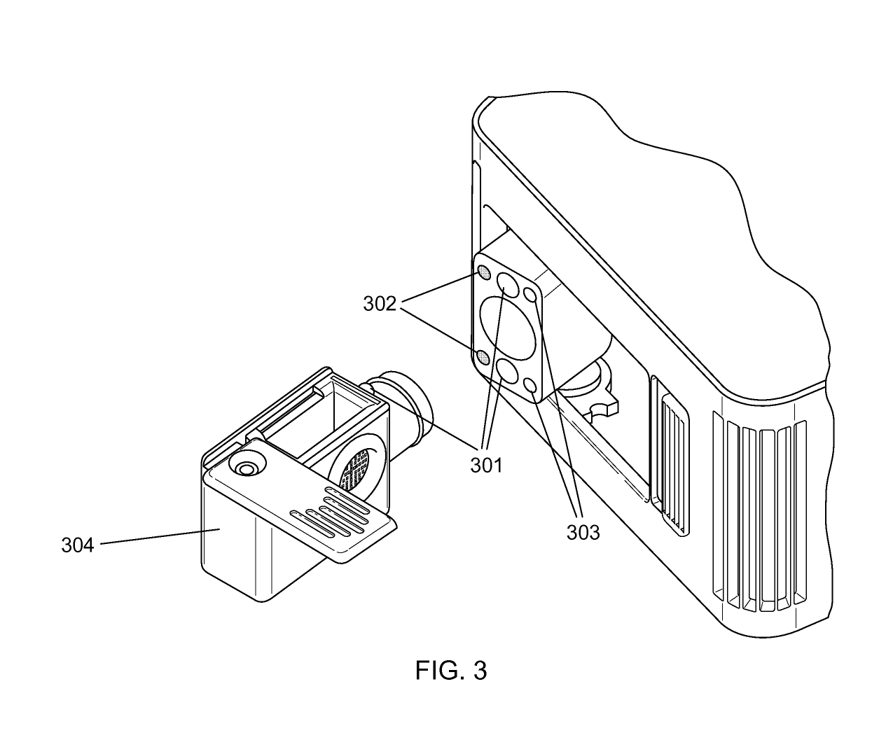

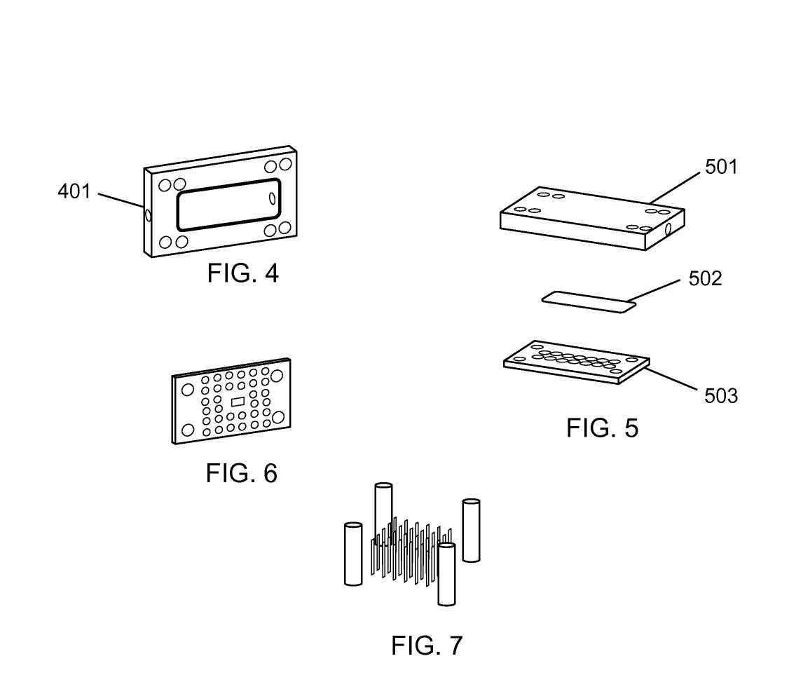

A sensor chamber with a sensor array is diagrammed in FIGS. 4 to 7. FIG. 4 shows an embodiment of the sensor chamber as a bottom view of a sensor chamber with an optional filter port 401. The filter can remove impurities from the volatile component in order to, for example, protect or increase the efficiency of the sensor array. FIG. 5 shows a sensor chamber 501, sealing gasket 502, and sensor array chip 503 in an exploded view. FIG. 6 shows a bottom view of the sensor array chip. FIG. 7 shows a pogo pin array to connect the electronics of the device to the sensor array chip. Other configurations are known to those skilled in the art.

The inventive device can further include a computing system. The computing system works to combine the responses of all of the sensors, which represents the input for the data treatment. This part of the instrument performs global fingerprint analysis and provides results and representations for interpretation. Moreover, the electronic nose results can be correlated to those obtained from other techniques (sensory panel, GC, GC/MS). Many data interpretation algorithms can be used for the analysis of results. These algorithms include, but are not limited to, principal component analysis (PCA), artificial neural network algorithms, multi-layer perception (MLP), generalized regression neural network (GRNN), fuzzy inference systems (FIS), self-organizing map (SOM), radial bias function (RBF), genetic algorithms (GAS), neuro-fuzzy systems (NFS), adaptive resonance theory (ART), partial least squares (PLS), multiple linear regression (MLR), principal component regression (PCR), discriminant function analysis (DFA), linear discriminant analysis (LDA), cluster analysis, and nearest neighbor.

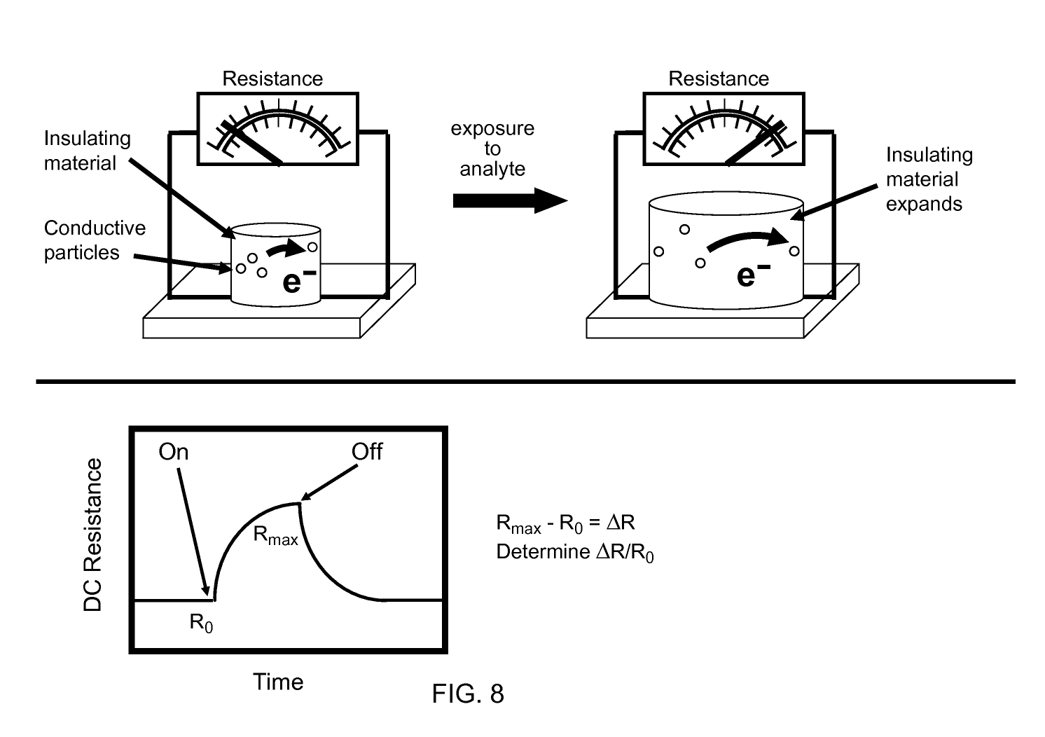

As disclosed above, sensor arrays can be used to differentiate analytes in a volatile component by the sensor array's unique response profile to the analyte. FIG. 8 shows the theory of operation of an exemplary sensor. As shown in FIG. 8, an exemplary sensor is a swellable material comprising an insulating material coated with conductive particles. In the absence of analyte, the sensor has a characteristic “resting” DC resistance, R₀. When the sensor is contacted with an analyte, the material swells characteristic to that analyte, causing the conductive particles to separate, and thereby causing a change in the resistance of the sensor characteristic for the analyte, Rₘₐₓ. Delta R is Rₘₐₓ−R₀. The normalized resistance difference ((Rₘₐₓ−R₀)/R₀)) or Delta R/R₀ is determined for the analyte. Those skilled in the art are aware of many other sensor types that operate similarly based on other physical properties.

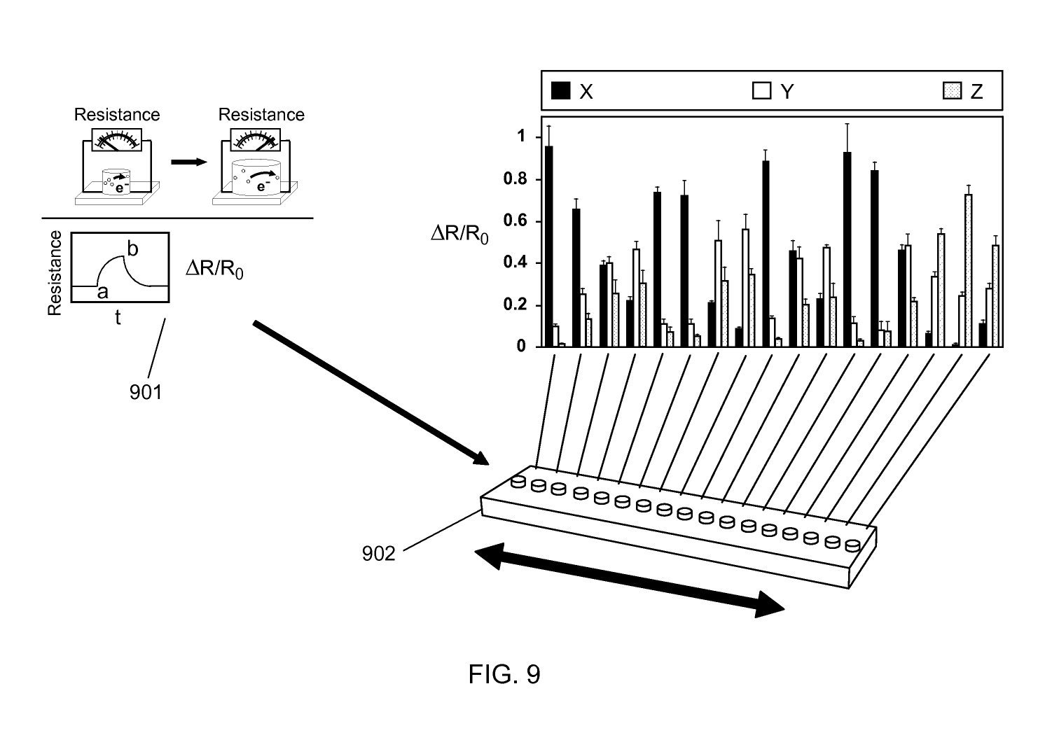

By using a plurality of sensors having different sensor films that react differently to different analytes, a profile of sensor responses can be generated characteristic for a specific analyte. An example is shown in FIG. 9. As shown, each individual sensor can be a unique sensor film coated on an electrode 901 in an array of sensors 902 that have different coatings. The Delta R/R₀ of each sensor can be different for different analytes. Therefore, a suitably produced sensor array 902 will result in a unique profile of Delta R/R₀ responses for each analyte. As shown in FIG. 9, analytes X, Y, and Z can be distinguished from each other by their unique sensor array response profiles. At 8-bit resolution, more than 10⁴⁸ distinct profiles are theoretically possible for a 16-sensor array. Those skilled in the art realize that larger arrays and more sensitive sensors can expand the potential for detecting distinct profiles.

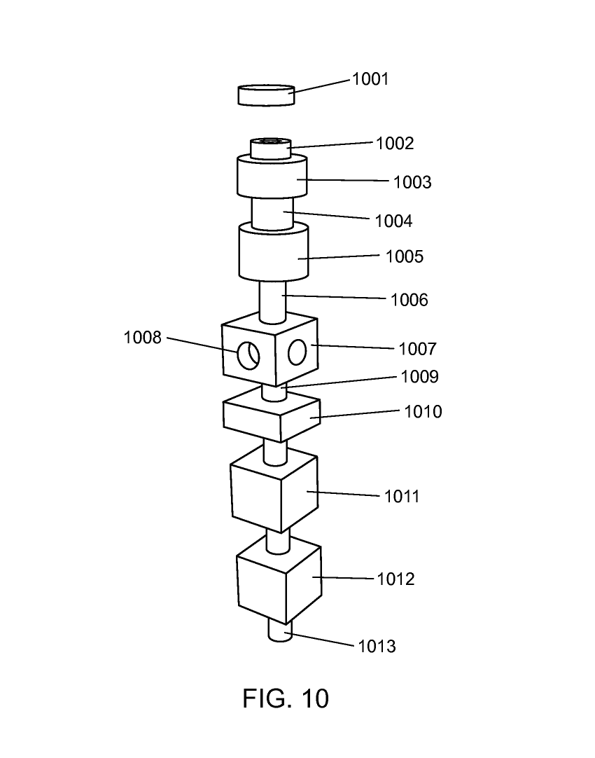

FIG. 10 shows another embodiment of the inventive device. The device includes a lid 1001 that covers sample reservoir 1002 surrounded by a heat chamber 1003. In one embodiment the heat chamber 1003 can consist of nichrome wire for heating and a thermo sensor (e.g. thermistor or thermocouple). It should be contemplated by one skilled in the art that the shape of the present invention is not limited to the shapes of the elements represented in FIG. 10. In a suitable embodiment the sample reservoir 1002 can be threaded for a screw top lid. The heat chamber 1003 can comprise any material suitable for thermal transfer such as metal, such as steel, or thermal paste. In one embodiment the heat chamber 1003 is aluminum. In an additional embodiment the lid could include a sonication apparatus for generating ultrasonic waves.

In a suitable embodiment of the present invention and not represented in the FIG. 10 drawing, the sample reservoir 1002 can be the end of the pipe 1004 where the lid 1001 fits over. The pipe can comprise any material suitable for thermal transfer such as metal, such as steel, or thermal paste. In one example of this embodiment, the pipe 1004 can be a 6 millimeter threaded brass rod with a hole drilled through the center.

In alternative embodiments, the heat chamber 1003 is connected to a heating element and a thermal sensor. In a suitable embodiment, thermal sensor comprises an infrared light sensor positioned sufficiently to measure chamber 1003 temperature. In a suitable embodiment, a heat chamber 1003 is coupled to a pipe 1004. In this embodiment (and not diagrammed in FIG. 10), a heating element could be a nichrome wire that is wrapped around the heat chamber 1003 which is coupled to pipe 1004.

In a suitable embodiment, the pipe 1004 can include material that absorbs vapors (e.g. a filter material). In a suitable embodiment, this material can comprise carbon. In another aspect, the pipe 1004 is coupled to a filter module 1010 that is a chamber that contains one or more filters. It can also be contemplated that a filter can be placed in the sample reservoir. Filters can be placed throughout the components in any order.

The pipe 1004 is connected to a coupler 1005 that consists of heat tolerant material having low thermal conductivity including, but not limited to, wood, rubber, or heat tolerant plastic such as Polytetrafluroethylene (PTFE). In one aspect, the coupler 1005 comprises Polyether ether ketone (PEEK).

The coupler 1005 is also connected to a proximal tube 1006. In this embodiment the coupler 1005 connects pipe 1004 to proximal tube 1006 that allows a volatile component to flow through. In one aspect, the coupler 1005 can include a filter. The filter can comprise carbon, polyester, polycarbonate, cellulose nitrate, mixed cellulose ester, nylon, polytetrafluoroethylene (PTFE), polypropylene, aluminum oxide, polyether sulfone (PES), or nitrocellulose. In another embodiment, the filter can be a high-efficiency particulate air or HEPA air filter. The skilled artisan recognizes that other filter material is available and that filters can be suitably placed optionally in and between each module and component. One example is to have carbon material to function as a filter for a gas.

The proximal tube 1006 is connected to a distribution module 1007 that connects multiple modules through connection ports 1008. The distribution module 1007 consists of one or more channels that can divert the flow of volatile components. The distribution module 1007 can also include valves for controlled diversion of flow. In an embodiment not shown in FIG. 10, a coupler that connects the pipe to tubing could be also a distributor for connecting one or more process modules.

A suitable embodiment shown in FIG. 10 comprises a detection module (referenced in FIG. 1) including: a filter module 1010, sensor module 1011, pump 1012 and an exhaust 1013. In this embodiment, the pump 1012 directs the flow of the volatile component from the heated sample reservoir 1002 to the sensor module 1011. In one embodiment, the sensor 1011 is connected to the pump via distal tube 1009. In another embodiment the pump 1012 can be directly connected to sensor module 1011. Any pump with a flow rate sufficient to advance the volatile component from the sample chamber module to the detection or distribution modules is suitable. Examples of suitable types of pumps include, but are not limited to, displacement, peristaltic, diaphragm and vacuum driven pumps. Such pumps include Flight Works gear pumps, Clark KPM Square Series miniature gas pumps and CurieJet micropumps. An exemplary pump is a piezoelectric driven pump such as the Microblower (Murata Electronics North America, Inc., Smyrna, Ga., USA).

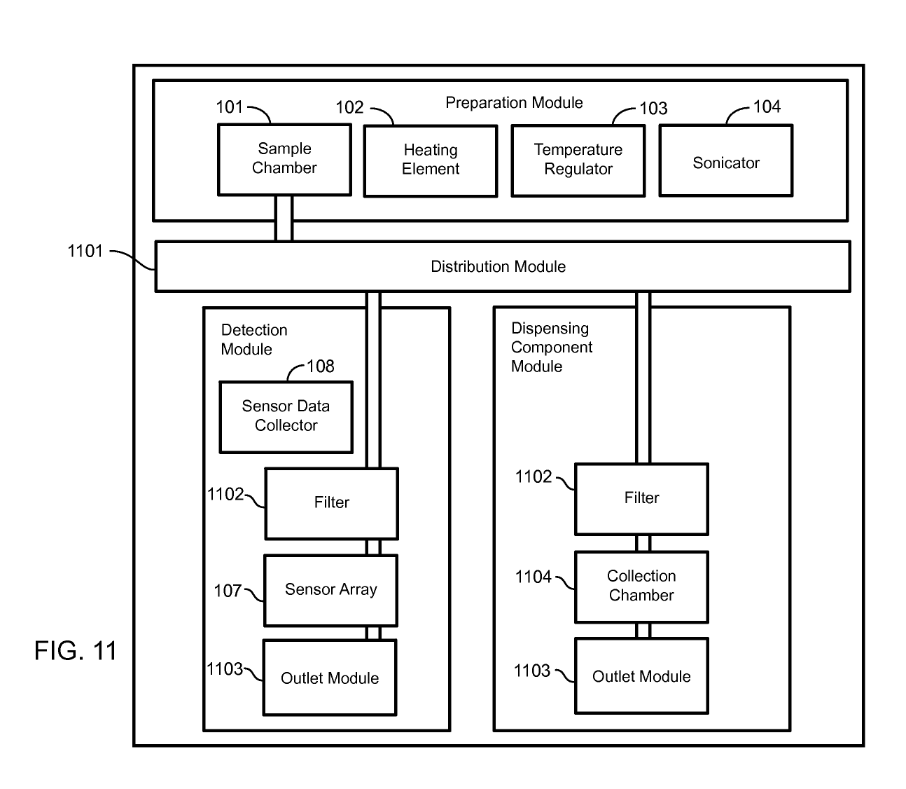

A more detailed overview of an embodiment of the device including a distribution module is shown in FIG. 11. The diagram shows a sample preparation module, distribution module, detection module and a dispensing component module. The sample preparation module is as described above. The distribution module 1101 directs the path of the volatile component. The detection module is as described above except that, in this embodiment it also optionally includes a filter 1102 and an outlet module 1103. The dispensing component module receives the volatile component for diversion, collection, administration or analysis of a volatile component or a subfraction thereof. In the embodiment shown, the dispensing component module optionally includes a filter 1102, an outlet module 1103, and a collection chamber 1104.

The distribution module 1101 directs the flow of volatile components or subfractions thereof to one or more connection ports. A connection port can be connected to a detection module or any other sensor device suitable for detecting an analyte. A connection port can be connected to a dispensing component module. The dispensing component module can include a filter 1102, a collection chamber 1104, or an outlet module 1103. The outlet module 1103 can be for the administration of a volatile component or subfraction thereof to a subject or to direct a volatile component or subfraction thereof to waste. The collection chamber 1104 can be for subsequent for analysis, concentration, administration, modification or disposal of the collected volatile component. A plurality of connection ports can be connected to any of the above in any combination or redundancy. For example, as shown in the Figure, the connection ports can be connected to a detection module and a dispensing component module. In other configurations, one or more detection modules and one or more sensor devices can be connected. In still other configurations, multiple dispensing component modules can be connected. In an embodiment, volatile component flow is controlled by a mobilization module 105. In an embodiment, the mobilization module comprises a pump. Numerous types of pumps are disclosed above. The pump or pumps can be computer controlled. A plurality of pumps can direct flow to designated outlet ports. The distribution module can include one or more valves. The valves can be manually or electrically controlled valves. Exemplary valves include, but are not limited to, rotary valves, solenoid valves, pinch valves and hydrogel valves. The valves can be computer controlled. Examples of suitable valves include a Parker X-valve, a Parker NEX-valve, a Valcor SV75 valve, a Lee Company LHD series of LVF series valve.

The detection module or dispensing component module can include one or more filters 1102. As disclosed above, the filter 1102 can comprise carbon, polyester, polycarbonate, cellulose nitrate, mixed cellulose ester, nylon, polytetrafluoroethylene (PTFE), polypropylene, aluminum oxide, polyether sulfone (PES), or nitrocellulose. In another embodiment, the filter 1102 can be a high-efficiency particulate air or HEPA air filter. The skilled artisan recognizes that other filter material is available and that filters can be suitably placed optionally in and between each module and component.

In one embodiment the outlet module 1103 can also include a mobilization module 105. In an embodiment, the mobilization module 105 comprises a pump or a plurality of pumps. The pump or pumps can be computer controlled. The outlet module 1103 can include one or more valves. The valves can be manually or electrically controlled valves. Exemplary valves are disclosed above. In a suitable embodiment the outlet module 1103 can have a direct flow back to the distribution module for enabling a series of synchronized loops for purification and detection of samples. In another embodiment the output module can be connected to a sample administration devices (e.g. inhaler) or waste output.

In a suitable embodiment the detection and dispensing component modules are in a parallel configuration. In an embodiment, the parallel configuration can be achieved using tubing in parallel. The tubing can be flexible to accommodate more designs. Exemplary tubing comprises rubber, polypropylene, or PEEK. The tubing can have any inner diameter suitable to allow mobilization of the volatile component. In an embodiment, the tubing can have an inner diameter range of 10 mm to 1 mm.

In another embodiment, the detection and dispensing component modules are in series. In other words, the volatile component that is exposed to the detection module is then allowed to flow to the dispensing component module.

In a suitable embodiment, the sample administration and sample detection paths are separated (in parallel). The advantage of this configuration is that the subject does not administer the volatile component or subfraction thereof where the volatile component has been exposed to the detection module.

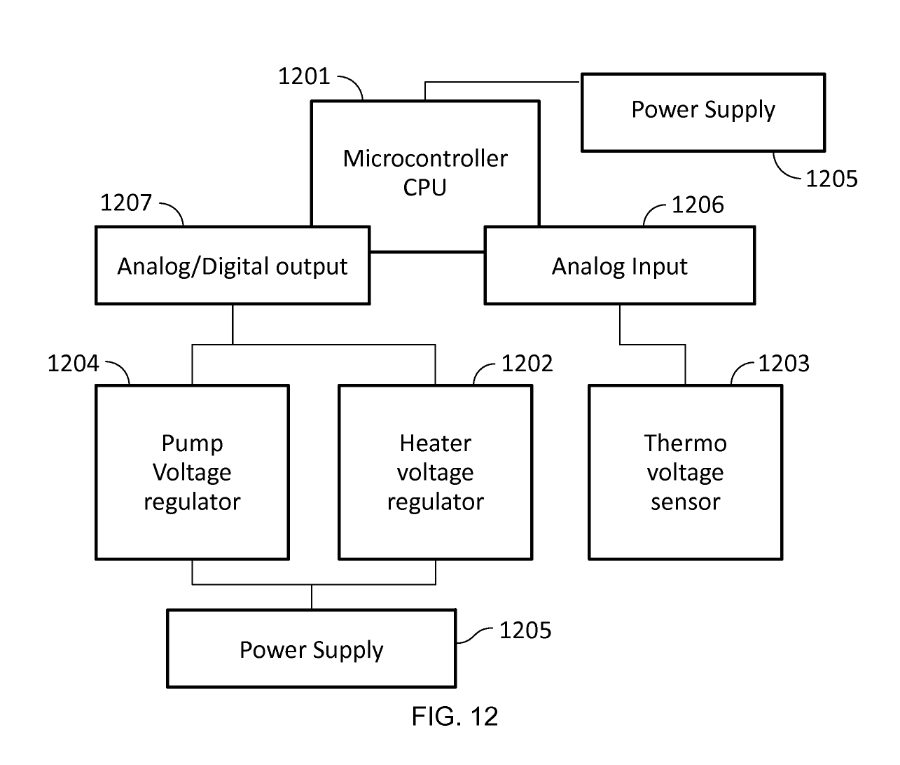

In suitable embodiments, the device further includes control electronics. FIG. 12 is a diagram of control electronics used as one embodiment of the present invention. In this embodiment a microcontroller or CPU 1201 is connected to a thermo voltage sensor 1202, heater voltage regulator 1203 and flow control voltage regulator 1204. Microcontrollers are known by skilled artisans. An exemplary microcontroller is an Arduino microcontroller (Ivrea, Italy). CPUs are known by skilled artisans. Exemplary CPUs are a Beagleboard (Texas Instruments, San Diego, USA), RaspberryPI (United Kingdom) or similar type of ARM computer.

In a suitable embodiment, the thermo voltage sensor 1203 is a voltage divider that is connected to the microcontroller 1201 that is connected to analog input 1206. The voltage sensor 1203 can be connected to a thermocouple or a thermistor. Other temperature sensors are known to those skilled in the art, including infra-red light sensing for example.

In another embodiment, a heater voltage regulator 1202 includes a power MOSFET transistor controlled by a pulse width modulating pin analog output or by a digital output pin 1207. In this embodiment, the MOSFET transistor of the heater voltage regulator 1202 can be connected to a separate power supply 1205. The separate power supply 1205 has sufficient power for heating. Suitable alternatives known by one skilled in the art include a similar transistor, relay or a h bridge rather than power MOSFET transistor.

In another embodiment, a flow control voltage regulator 1204 is used for driving a pump. For example, the voltage regulator can be a wave form generator and amplifier. Alternatively, a waveform can be generated using a microcontroller and can amplify the waveform using an op-amp amplifier or a boost regulator. Other waveform generator and amplifier systems are known to the skilled artisan. In an embodiment, a step up transformer to boost converter, and op-amp, can be included. In another embodiment, the flow control voltage regulator 1204 can be used to switch valves such as a rotary valve. In another embodiment, the flow control voltage regular 1204 can be used to actuate a solenoid for restricting flow or allowing flow. Flow control regulators are known to skilled artisans.

In another embodiment, the control electronics optionally includes a operator signal generation apparatus, such as an LED indicator, for communicating a device related event to the operator of the device. The signal generation apparatus can provide a light, a tone, a vibration and the like. In one embodiment, an LED can be used to indicate that the sample is being heated. In another embodiment, an LED can be used to indicate when to switch valves. In another embodiment an LED can be used when to signal to the subject when to inhale volatile components or subfractions thereof.

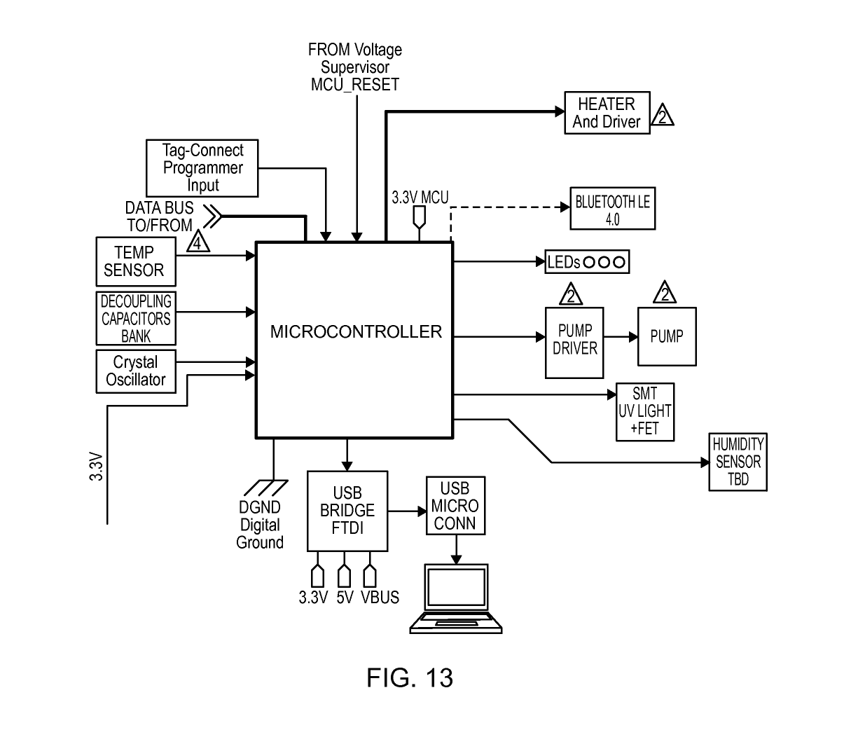

FIG. 13 shows another embodiment of the control electronics.

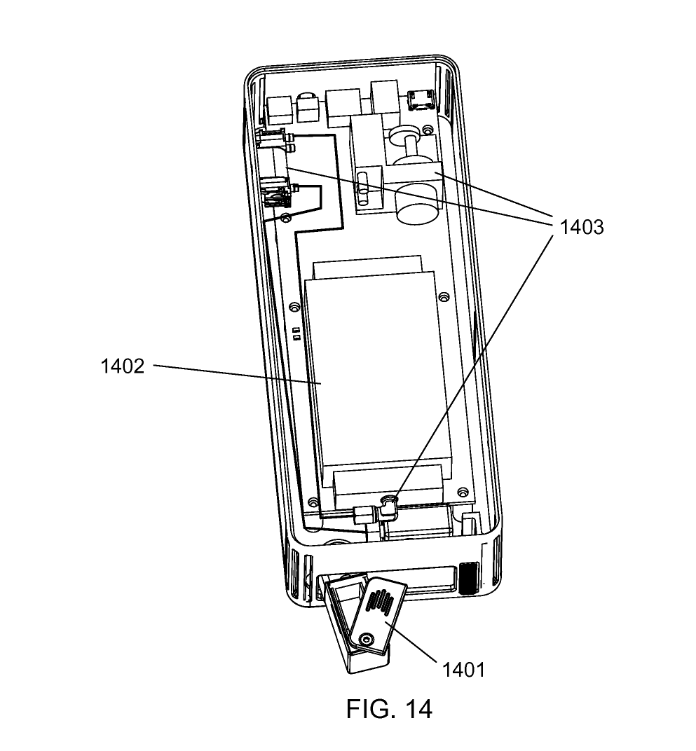

FIG. 14 shows a perspective view illustration of an embodiment of the inventive device. Shown therein is the sample preparation module 1401, the detection module 1402 and the distribution module 1403.

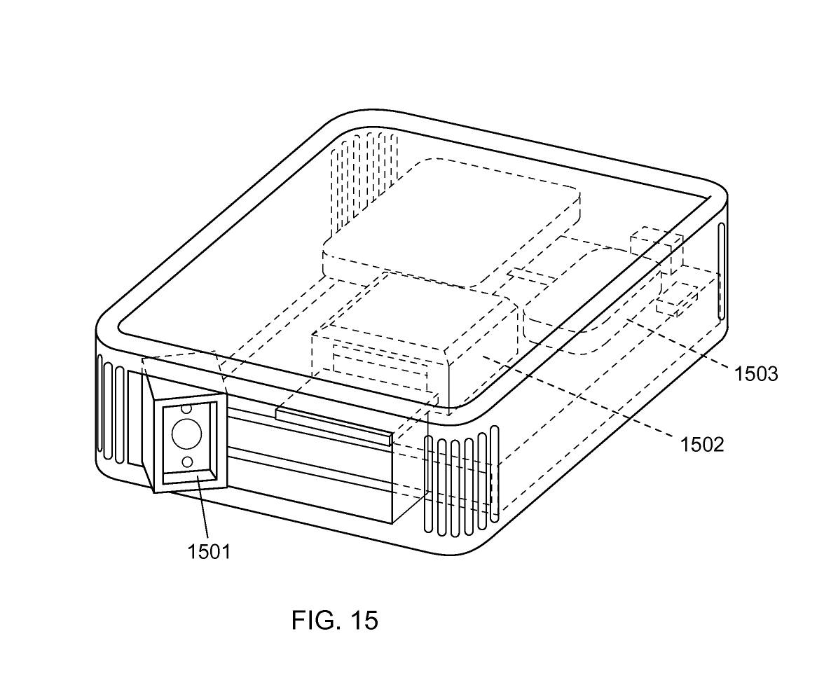

FIG. 15 shows a perspective view illustration of another embodiment of the inventive device. In this view, shown is the connector for the sample preparation module 1501, the detection module 1502 and the distribution module 1503 that includes a pump.

Provided herein are methods related to the use of the disclosed devices for the detection and delivery of sample volatile components and subfractions thereof. The sample can be of any origin as disclosed above.

Provided herein are methods of analyte detection by (a) volatilizing a sample resulting in a volatile component, (b) separating the volatile component from the remaining sample, and (c) contacting the volatile component with a sensor array, wherein the sensor array detects the analyte in the volatile component when the analyte is present in sufficient concentration. Examples of volatilizing include, but are not limited to, heating, sonication, stirring, grinding and mechanical shearing. In suitable embodiments, the sample temperature can be modulated to selectively volatilize at least one analyte. In suitable embodiments, the sample is heated and sonicated. In suitable embodiments, the sample is heated and ground. Suitable apparatuses for volatilization are disclosed above.

In the case where the sample is Cannabis, different volatile components volatilize at different temperatures. For example, see the Table below:

TABLE 1
Heat profiles of Cannabinoids, Terpenoids and Flavanoids

| | Vaporization Temperature | |
|---|---|---|
| Cannabinoid | | |
| Tetrahydrocannabivarin (THCV) | 137.6° | C. |
| Delta-8-tetrahydrocannabinol (delta-8-THC) | 144.5° | C. |
| Delta-9-tetrahydrocannabinol (THC) | 149.3° | C. |
| Cannabichromene (CBC) | 174.2° | C. |
| Cannabidiol (CBD) | 206.3° | C. |
| Cannabigerol (CBG) | 207.2° | C. |
| Cannabinol (CBN) | 212.7° | C. |
| Terpenoid | | |
| Linalool | 198° | C. |
| d-Limonene | 177° | C. |
| Myrcene | 166° | C. |
| alpha-Pinene | 156° | C. |
| Beta-Caryophyllene | 119° | C. |
| Flavonoid | | |
| β-sitosterol | 134° | C. |
| Apigenin | 178° | C. |
| Cannflavin A | 182° | C. |
| Quercetin | 250° | C. |

Consequently, the sample can be heated to a temperature optimal for release of a certain constituent into the volatile component that does not favor other constituents. For example, a sample of Cannabis can be heated to 150° C. and favor the release of Tetrahydrocannabivarin (THCV), Delta-8-tetrahydrocannabinol (delta-8-THC), Delta-9-tetrahydrocannabinol (THC), but not the other cannabanoids listed in the table.

The volatile component is separated from the remaining sample. The volatile component can flow from a sample module to a detection module in fluid communication such as disclosed in the devices above. Such transfer can be aided by a mobilization module such as disclosed above. The separation of volatile component from the remaining sample can include filtration of the volatile component. Various filter types and media are disclosed above.

The volatile component is then contacted with a sensor array. The sensor array detects at least one analyte if present. Sensor arrays are disclosed above. The sensor array can be connected to a microcontroller, a memory and an input/output device. The system can further include a CPU. The sample's sensor array profile is compared to profiles of known analytes and the type of analyte and amount is determined and this information is sent to the input/output device. The subject can be alerted to the analyte type and amount.

Also provided herein are methods for regulating delivery of a volatile component by (a) volatilizing a sample resulting in a volatile component, (b) separating the volatile component from the remaining sample, (c) contacting the volatile component with a sensor array, wherein the sensor array detects an analyte in the volatile component when the analyte is present in sufficient concentration, and (d) regulating delivery of the volatile component based on a preset level of analyte delivery. In suitable embodiments, delivery is to a subject.

The volatilizing, separating and contacting steps are as disclosed above. The volatile component is then delivered subject to control either by the subject or by automation. In a suitable embodiment, the volatile component flows to a distribution module. Distribution modules are disclosed above. The distribution module can include valves and outlet connectors that divert the flow of volatile component to an outlet port that directs the volatile component to the subject or to waste. Flow can be directed by the subject or by automation to regulate the delivery of the volatile component to the subject. For example, the subject can be alerted to the delivery of a threshold level of an analyte and divert the flow of volatile component to a collection chamber or to waste. As another example, the flow can be diverted automatically (without intervention from the subject) to waste or to a collection chamber when a threshold level of an analyte in the volatile component has been delivered. In other embodiments, the volatile component is delivered in parallel to the detector and to other outlet connectors so that the volatile component consumed by the subject is separated from volatile component that is contacted with the sensor array.

In an embodiment, the volatilizing of the sample is by heating and the temperature is modulated to selectively volatilize at least one analyte. Apparatuses for incremental heating are disclosed above. For example, by applying a heat gradient to a sample, different analytes will be preferentially released from the sample at different times. Consequently, by modulating the temperature (or causing differential release of analytes into the volatile component of the sample by other volatilization methods), the volatile component can have an increased or decreased concentration of a specific analyte.

In suitable embodiments, the method further includes (a) a filtration step between volatilizing the sample and contacting the volatile component with a sensor array or (b) a filtration step between the sensor array and the delivery of the volatile component. Various filter types and media are disclosed above.

In another embodiment, the volatile component is collected before delivery. For example, the volatile component can be collected in a collection chamber as described for the devices disclosed above. The volatile component can be concentrated before delivery. Concentration can be achieved by condensation of the volatile component. For example, the volatile can be directed to a collection chamber that is either at a temperature, or includes a substance, that is below the dew point of the volatile component or subfractions thereof thereby converting the volatile component or subfraction thereof to liquid. The liquid can then be diverted for delivery. The delivery can be to a subject or a waste receptacle. In suitable embodiments, the delivery is to a detection module or some other sensor.

In suitable embodiments, the subject can adjust the preset level of analyte. For example, the subject may wish to adjust the level of analyte to be delivered for dosage control of a drug or supplement in the volatile component. For example, the subject consuming a recommended level of analyte, in consultation with a medical professional, may want to reduce the amount of analyte delivered to reduce unwanted side effects. The subject can adjust the preset level to a lower level of analyte for delivery. In other embodiments, the preset level is automated so that adjustment by the subject is unnecessary.

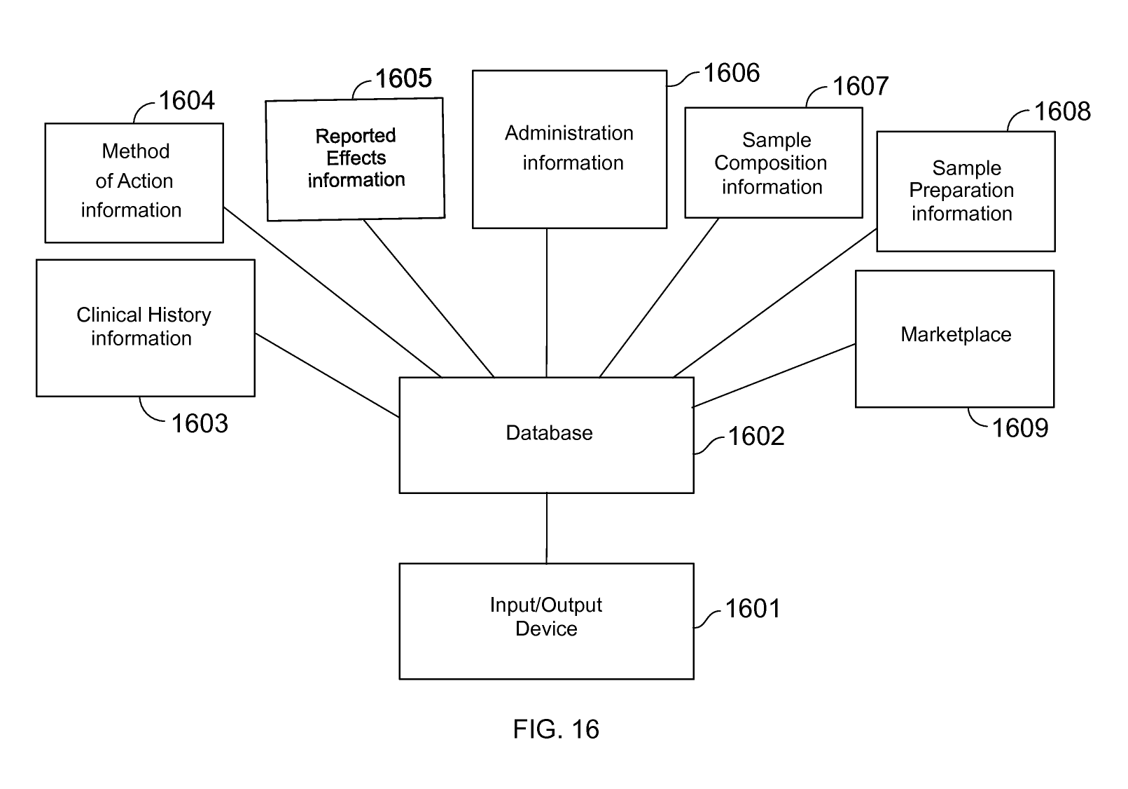

The subject can be provided sample-related information to assist the subject in preparing the sample, detecting the analyte, or adjusting the amount of analyte to be delivered. The sample-related information can be sensor information, clinical history information, method of action information, reported effects information, administration information, sample composition information, sample preparation information, and marketplace information. Aspects of this information are diagrammed in FIG. 16. The end user accesses this information through an input/output device 1601 and that is stored in a database 1602.

The sample-related information can include clinical history information 1603. This category can include subject derived data wherein the subject provides medical history data. Medical history data can include diseases and conditions, dates of onset, past treatments. Clinical history information can also be referenced information about the history of the medical condition and how it is currently treated. The source of referenced information can include but is not limited to scientific journals, medical textbooks, wikipedia sourced documentation and clinical handbooks. Clinical history information can be derived from social media sources such as where people communicate how they are coping with similar medical conditions.

The sample-related information can be method of action information 1604. Method of action information can include the biochemical mechanism, indications, contraindications, and dosing regimens. The information can be collected from referenced information. The source of referenced information can include but is not limited to scientific journals, medical textbooks, wikipedia sourced documentation and clinical handbooks. The source of referenced information can include can be from social media sources.

The sample-related information can include reported effects information 1605. Reported effects information can both be end user derived information and community derived information (e.g. social media derived sources). Reported effects can include subjective health improvement post-treatment, potential side effects and suitable alternatives to address the medical condition.

The sample-related information can include administration information 1606. This information includes details about dosing protocols which can be derived from referenced information or community derived information. In a suitable embodiment, the administration process is automated. Automation can control one or more of sample preparation, detection and delivery and the like.

The sample-related information can include sample composition information 1607. This information can be derived from the devices provided herein, referenced information and community derived information. In a suitable embodiment, a sample is characterized by a device of the invention that prepares the drug and characterizes its composition during or after the preparation process. This data can be collected and distributed to other end users and medical service providers. In an additional embodiment, the subject can run applications that are tailored to analyze the drugs compositions from the network. In this embodiment the application would be a computer that can operate the analysis device or statistically analyze raw data derived from the instrument.

The sample-related information can include sample preparation information 1608. This information can be from referenced sources or can be community derived. The preparation process can be manually performed by the subject or it can be performed in an automated process. In a suitable embodiment, the automated process can be controlled by a computer program.

The sample-related information can include a marketplace for distribution of healthcare related data 1609. This marketplace can consist of healthcare product and service providers and can provide solutions for healthcare management, drug preparation tools, data analysis tools and consumables for scientific instrumentation and materials for preparing and dosing therapeutics.

Also provided herein are methods for regulating delivery of a subfraction of a volatile component including (a) volatilizing a sample resulting in a volatile component, (b) separating the volatile component from the remaining sample, (c) contacting the volatile component with a sensor array, wherein the sensor array detects an analyte in the volatile component when the analyte is present in sufficient concentration, (d) fractionating the volatile component into a plurality of subfractions, and (e) regulating delivery of at least one subfraction of the volatile component based on a preset level of analyte delivery. In suitable embodiments, the delivery is to a subject.

The volatilizing, separating and contacting steps are as disclosed above. The volatile component is then subjected to fractionation to concentrate an analyte in a subfraction of the volatile component. In a suitable embodiment, the volatile component flows to a distribution module that includes one or more control valves and one or more connection ports. In some embodiments, the analyte is preferentially released from the sample into the volatile component in a time-dependent manner. For example, the analyte may only be released after some period of time upon application of a constant temperature. In other embodiments, the analyte is preferentially released from the sample into the volatile component at a specific application of a volatilization force. For example, a temperature gradient can be applied to a sample and an analyte may be preferentially released from the sample into the volatile component at a specific temperature range. The temperature gradient can be, for example, a linear or step gradient. Consequently, in an embodiment, the method includes fractionating the volatile component into subfractions by heating and the temperature is modulated to selectively volatilize at least one analyte. The distribution module can be controlled, for example, to divert the volatile component to waste when the analyte is not present in the volatile component and then to a collection chamber or to a port to a subject when the analyte is preferentially released. In other embodiments, the analyte is fractionated for the purpose of sending the analyte to waste, such as where the analyte is a contaminant in the sample.

In suitable embodiments, the method further includes (a) a filtration step between volatilizing the sample and contacting the volatile component with a sensor array or (b) a filtration step between the sensor array and the delivery of the volatile component. Delivery can be to a subject. The filter can be before or after the distribution module. Various filter types and media are disclosed above.

In another embodiment, the volatile component is collected before delivery. For example, the volatile component can be collected in a collection chamber as described for the devices disclosed above. The volatile component can be concentrated before delivery. The delivery can be to a subject or a waste receptacle. In suitable embodiments, the delivery is to a detection module or some other sensor.

In suitable embodiments, the subject can adjust the preset level of analyte. Adjusting the level of analyte delivered is disclosed above.

As an example, a subject can receive a pre-programmed dosage of each cannabinoid and/or terpenoid in a Cannabis sample, using the device configured to divert undesired volatile component subfractions to exhaust and deliver desired volatile component subfractions at desired quantities to the subject. For example, the sample preparation module is heated and maintained at various pre-programmed temperatures. The aromatic terpenoids begin to vaporize at 126.0° C. (258.8° F.), but the more bio-active tetrahydrocannabinol (THC), cannabidiol (CBD) and cannabinol (CBN) do not vaporize until near their respective boiling points: THC 149.3° C. (300.7° F.), CBD 206.3° C. (403.3° F.), and CBN 212.7° C. (414.9° F.). Consequently, by heating the Cannabis sample in steps, the terpenoids can be diverted to waste at the lower temperature. At the higher temperature, the more bioactive analytes can be collected and/or delivered to the subject thereby concentrations of each component of interest are delivered in the volatile component to achieve the therapeutic effect desired.

The subject can be provided sample-related information to assist the subject in preparing the sample, detecting the analyte, or adjusting the amount of analyte to be delivered. Embodiments of sample-related information are disclosed above.

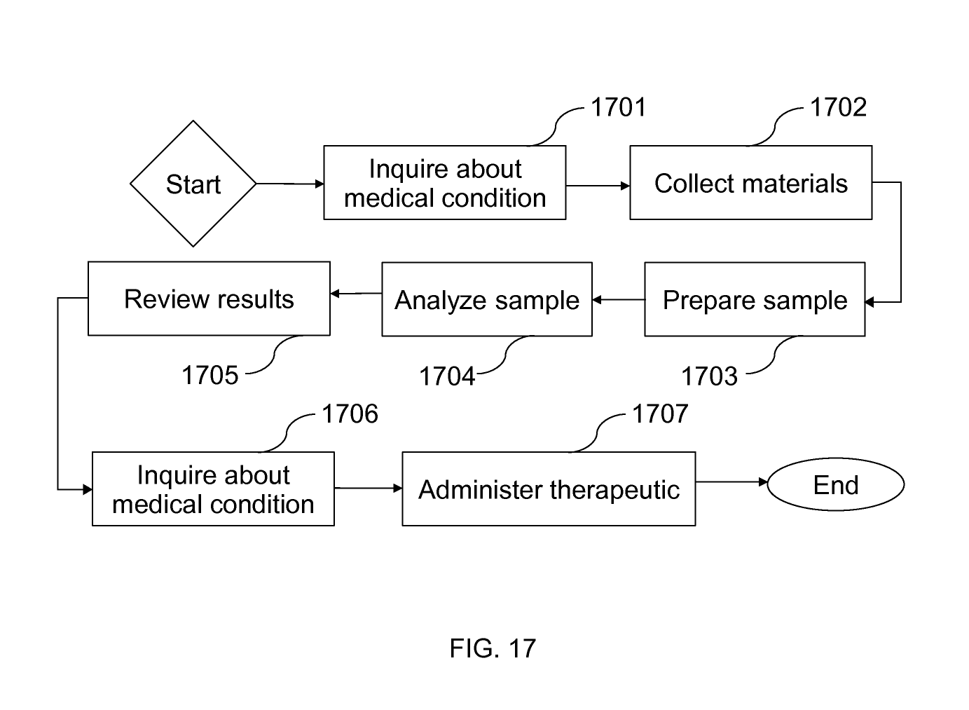

A representative example work flow is shown in FIG. 17. A user inquires about a particular medical condition 1701—this can be done by accessing referenced information. The source of referenced information can include, but is not limited to, scientific journals, medical textbooks, wikipedia sourced documentation, clinical handbooks and social media sources. Materials are collected 1702 and used for preparing a therapeutic sample 1703. The preparation process can include but is not limited to mixing, heating, pressurizing, drying, grinding and sonicating. The sample is then analyzed 1704 using a device of the invention.

The experimental results are reviewed 1705. In this embodiment the user may implement a specialized program that was acquired from a search into the referenced information. For example, a user can download a computer program that runs an application that prepares cannabis for intake. In another embodiment the results are compared to other experimental data. After reviewing the results, the appropriate dosage parameters are selected 1706.

In one embodiment the dosing phase 1707 can be inhaling chemicals. In another embodiment the administration 1707 is done by swallowing chemicals. After chemicals are ingested, the effects are tracked. The type of tracking can include, but is not limited to, device generated data, end user reported information and environmental related data.

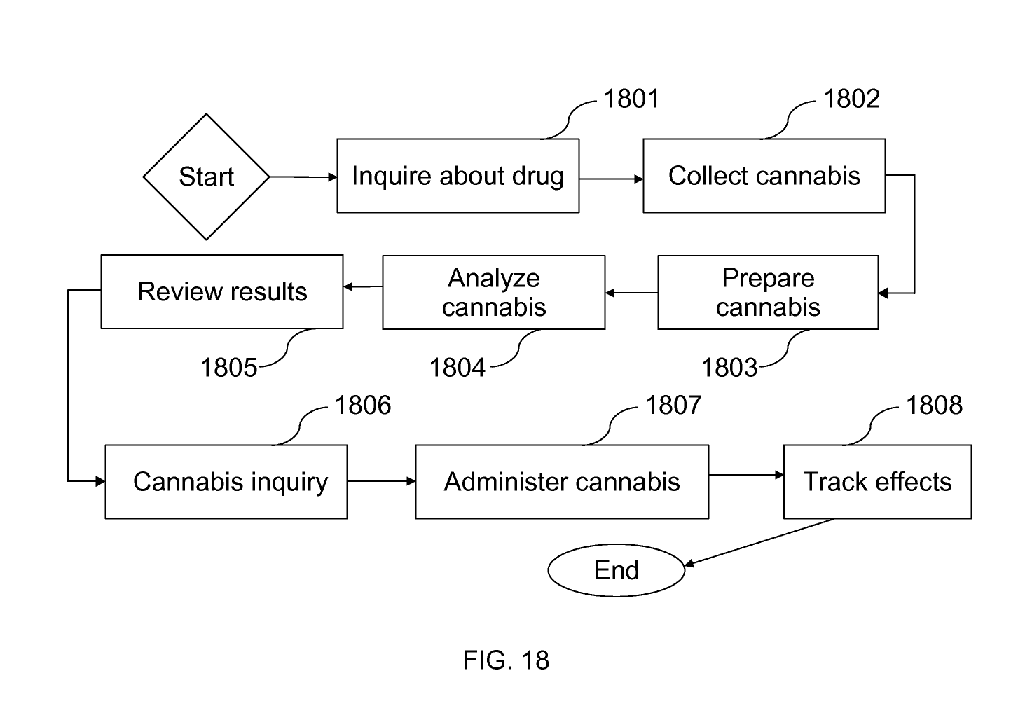

An example of the present invention is to prepare and consume cannabis derived consumables for the treatment of chemotherapy side effects as shown in FIG. 18. The patient consults with a medical professional 1801 and is advised to consume cannabis. The patient collects cannabis 1802 and prepares a sample of cannabis 1803.

In one embodiment, before consumption the cannabis sample is analyzed 1804. The results are collected and reviewed 1805. This data collection can be combined with cannabis related information collected over the internet 1806. This information is used to assess how the dosing will be conducted 1807. After dosing the effects are tracked 1008.

### Example 1

### Water Purity Detection

A water sample is introduced in sample chamber 101. The temperature regulator 103 is set to the desired temperature, which heats the sample using the heating element 102. Volatile components are released from the water sample. The volatile components are moved by the mobilization module 105 to the detection module. The sensor array 107 elements interact with analytes in the volatile component of the sample. Profiles characteristic of sensor array 107 responses for specific analytes are compared to sensor array responses obtained from the volatile component using a computing system. The computing system displays to the user data indicating the amount of each analyte. The user decides whether the water is safe for consumption.

### Example 2

### Pesticide and Pathogen Detection

### Detection of Pesticide

Detection of B-Myrcene and 2,4-D (2,4-Dichlorophenoxyacetic acid) compounds by various sensor arrays was determined using a multitude of functionalization materials and deposition patterns.

Sensor Functionalization:

The functionalized sensing materials tested were:

| | |
|---|---|
| 1. | SWNTs (98% semiconducting SWNTs (NanoIntegris Inc.) |
| 2. | SWNTs-Te nanostructures: Te nanoparticles; Te rice nanostructure; |
| | Te nano feather |
| 3. | SWNTs-Au NPs |
| 4. | SWNTs-Pd NPs |
| 5. | SWNTs-SnO2 NPs |
| 6. | SWNTs-SnO2 NPs-Pt NPs |
| 7. | PANI-CSA thin film |
| 8. | PEDOT-PSS thin film |
| 9. | SWNTs-Porphyrin based compounds: ZnOEP, FeOEP, FeTPP, NiPh, |
| | CuPh, TPP, RuTPP, NiTPP, CoTPP, MnTPP, OEP, RuOEP |

Solid pesticide compound (1 gram) was heated to 160 C (0.2 mm Hg) and the volatile component (vapor) was collected and introduced to each type of functionalized sensor.

Both compounds were detected with selectivity and sensitivity by the Te nanoparticles.

### Pesticide and Pathogen Detection in Cannabis

A Cannabis sample is introduced in sample chamber 101. The temperature regulator 103 is set to the desired temperature, which heats the sample using the heating element 102. Volatile components are released from the Cannabis sample. Among the volatile components are various pesticides and pathogens. The volatile components are moved by the mobilization module 105 to the detection module. The sensor array 107 elements interact with analytes in the volatile component of the sample. Profiles characteristic of sensor array 107 responses for pesticides and pathogens are compared to sensor array responses obtained from the volatile component using a computing system. The computing system displays to the user data indicating the amount of each pesticide and pathogen, if present. The user decides whether the product is safe for consumption.

### Example 3

### Air Quality Detection

An air sample is introduced in sample chamber 101. The temperature regulator 103 is set to the desired temperature, which heats the sample using the heating element 102. Volatile components are released from the air sample. Among the volatile components are various pollutants. The volatile components are moved by the mobilization module 105 to the detection module. The sensor array 107 elements interact with pollutants in the volatile component of the sample. Profiles characteristic of sensor array 107 responses for pollutants are compared to sensor array responses obtained from the volatile component using a computing system. The computing system displays to the user data indicating the amount of each pollutant, if present.

### Example 4

### Allergen Detection

A food sample is introduced in sample chamber 101. The temperature regulator 103 is set to the desired temperature, which heats the sample using the heating element 102. Volatile components are released from the food sample. Among the volatile components are various allergens. The volatile components are moved by the mobilization module 105 to the detection module. The sensor array 107 elements interact with allergens in the volatile component of the sample. Profiles characteristic of sensor array 107 responses for allergens are compared to sensor array responses obtained from the volatile component using a computing system. The computing system displays to the user data indicating the amount of each allergen, if present, thereby indicating the potential of the sample for eliciting an allergic reaction to a subject sensitive to the allergen.

### Example 5

### Crop Ripeness Score

A plant sample is introduced in sample chamber 101. The temperature regulator 103 is set to the desired temperature, which heats the sample using the heating element 102. Volatile components are released from the plant sample. Among the volatile components are various organic markers relating to ripeness. The volatile components are moved by the mobilization module 105 to the detection module. The sensor array 107 elements interact with organic markers in the volatile component of the sample. Profiles characteristic of sensor array 107 responses for organic markers are compared to sensor array responses obtained from the volatile component using a computing system. The computing system displays to the user data indicating the amount of each organic markers, if present, thereby indicating the ripeness of the plant sample.

### Example 6

### Cancer Marker Detection in Exhaled Breath

An exhaled breath sample from a subject is introduced in sample chamber 101. The temperature regulator 103 is set to the desired temperature, which heats the sample using the heating element 102. Volatile components are released from the exhaled breath sample. Among the volatile components are various biologic markers relating to cancer. The volatile components are moved by the mobilization module 105 to the detection module. The sensor array 107 elements interact with biologic markers in the volatile component of the sample. Profiles characteristic of sensor array 107 responses for biologic markers are compared to sensor array responses obtained from the volatile component using a computing system. The computing system displays to the user data indicating the amount of each biologic marker, if present, the presence indicating whether the subject is likely to have the cancer related to the biologic marker.

### Example 7

### Response Sensitivity of Sensor to Volatile Component

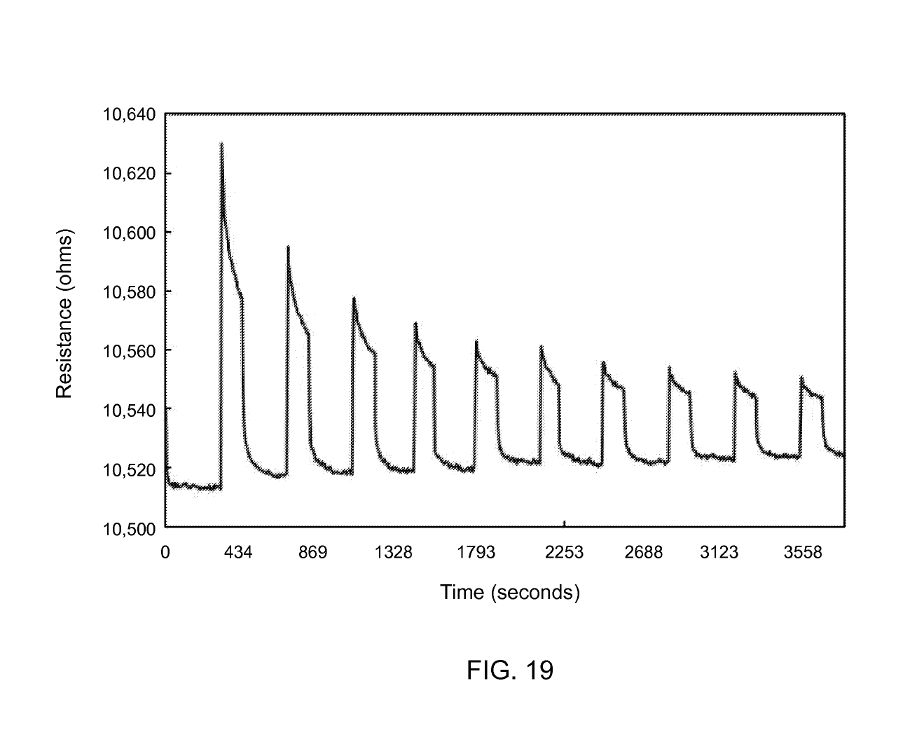

A sample of an organic compound was placed in the sample preparation module. FIG. 19 shows the response of a sensor channel 2 (resistance) to repeated exposure (ten times) of the volatile component of the sample. Although the magnitude of the response attenuates over repeated exposures, the sensor responds to the presence of analyte at each exposure.

### Example 8

### Distinct Sensor Array Profiles of Different Strains of Cannabis

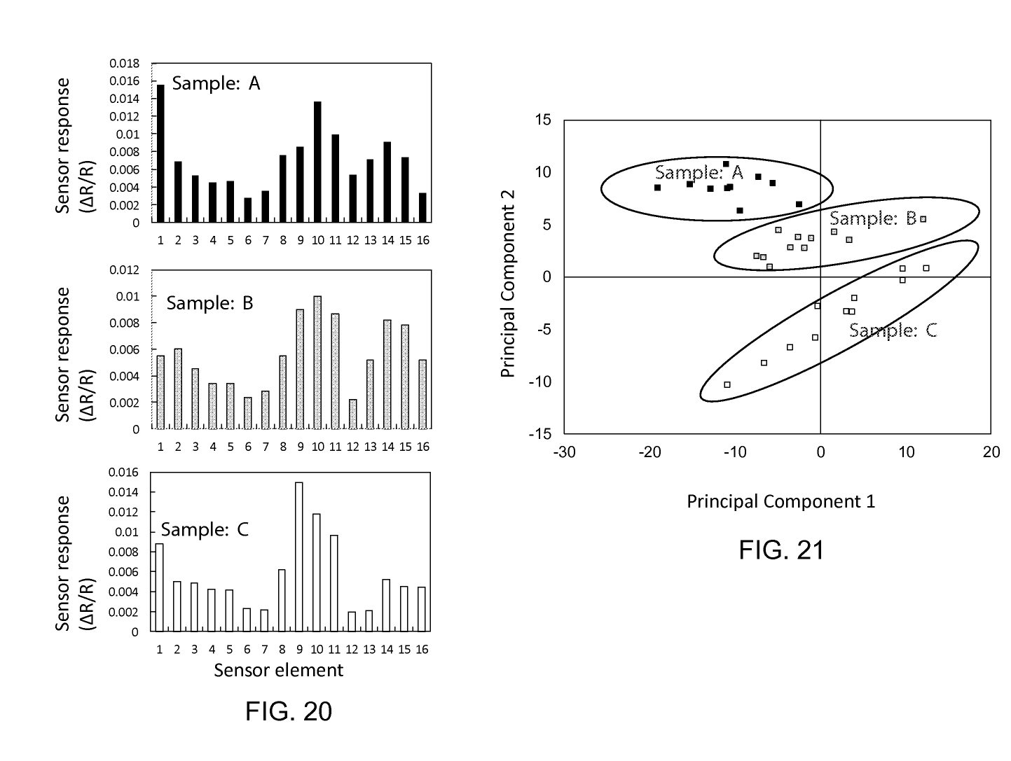

Three different strains of Cannabis preparations were prepared for analysis of a Cannabis sample was placed in the sample preparation module, the lid was closed and the module inserted into the device. The volatile component of the sample was and the relative resistance for each sensor in the array was measured. FIG. 20 shows the relative resistance change for each sensor in the array for each of the samples (A, B and C). The sensor array profiles are distinct from one another and therefore the profile can be used as a “fingerprint” for the interrogated strain. FIG. 21 shows principal component analysis for each sensor array profile further demonstrating the unique profile for each Cannabis strain type.

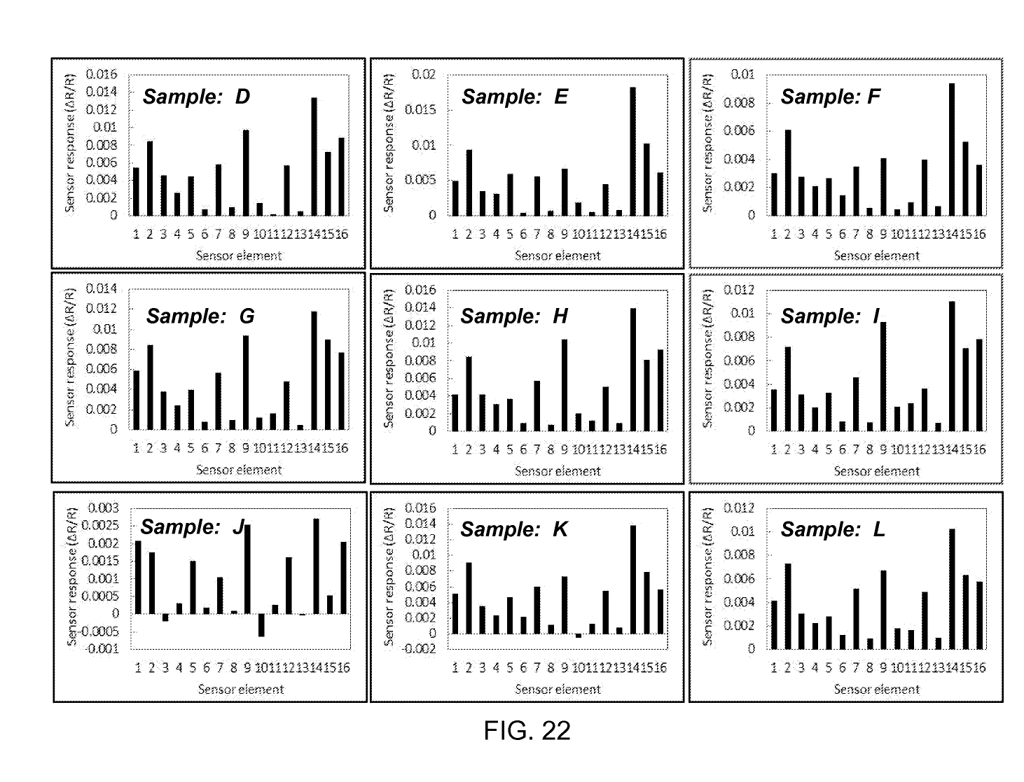

FIG. 22 shows sensor array profiles using a different sensor array than that used for FIGS. 21 and 22. In FIG. 22, 9 different Cannabis strains are profiled and shown to be distinct.

### Example 9

### Linear Sensor Response

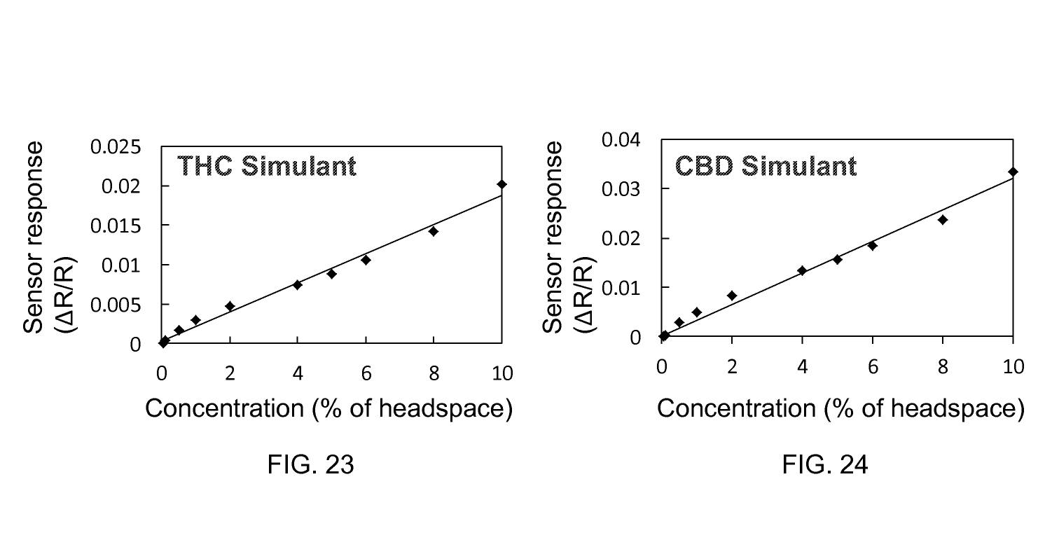

The signal amplitude of the relative resistance of a sensor was determined for two different Cannabis-related analytes. A THC simulant was placed in the sample preparation module, the lid was closed and the module inserted into the device. The relative resistance for each sensor in the array was measured. FIG. 23 shows a linear increase in relative resistance of one of the sensors as a function of THC simulant concentration. The experiment was repeated with a CBD simulant. As shown in FIG. 24, a linear increase in the relative resistance of one of the sensors as a function of CBD simulant concentration was observed. Consequently, the inventive device can provide measurement of concentration of analyte as well as identity.

### Example 10

### User Interface for Cannabis Analysis

The user interface for the device allows the user to coordinate use of the device for therapies. The interface can be displayed on a computer, such as a laptop or tablet, or a smartphone. For example, the following figures show a user interface displayed on a smartphone (an Apple® iPhone®).

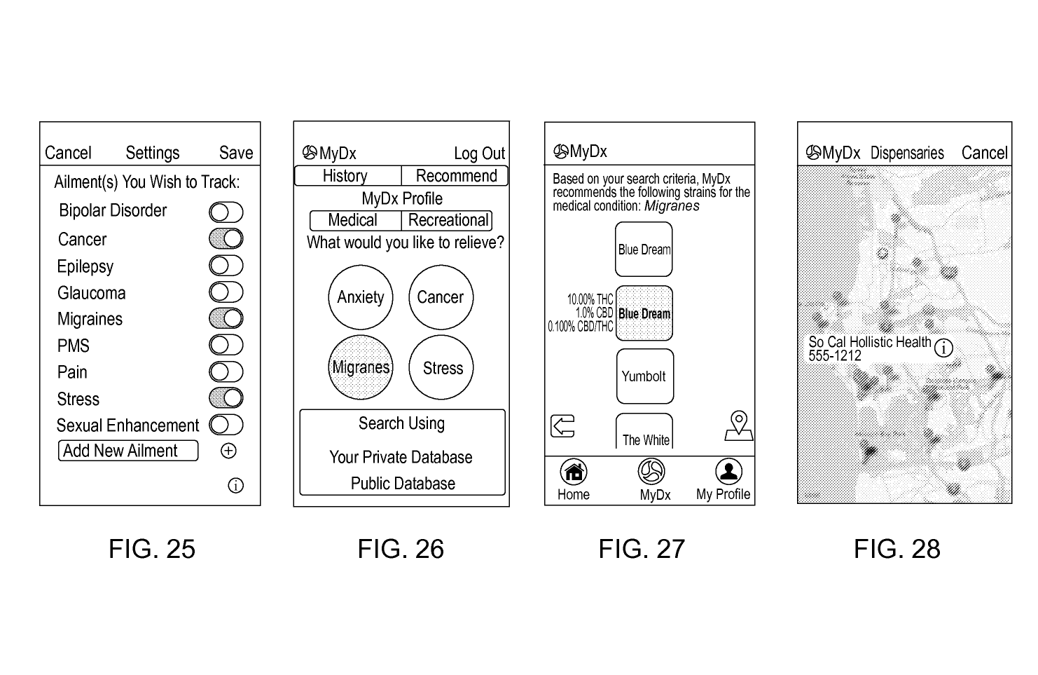

Profile mode. The user selects the diseases or disorders he or she wishes to track (FIG. 25). The user can select among diseases or disorders in psychology, oncology, skeletomuscular, etc. The user selects among diseases or disorders he or she wishes to treat, e.g. anxiety, cancer, migraines, stress, etc. in the medical tab (FIG. 26). Or the user selects among the recreational benefits being sought (FIG. 26). The user selects condition named migraines. The user is presented with a list of Cannabis strains known to be suitable for treating the condition (FIG. 27). The user selects one of the strains and is provided information regarding the relative levels of active components for the selected strain (FIG. 27). The user touches a map icon and is presented a map of nearby dispensaries that stock the selected strain (FIG. 28).

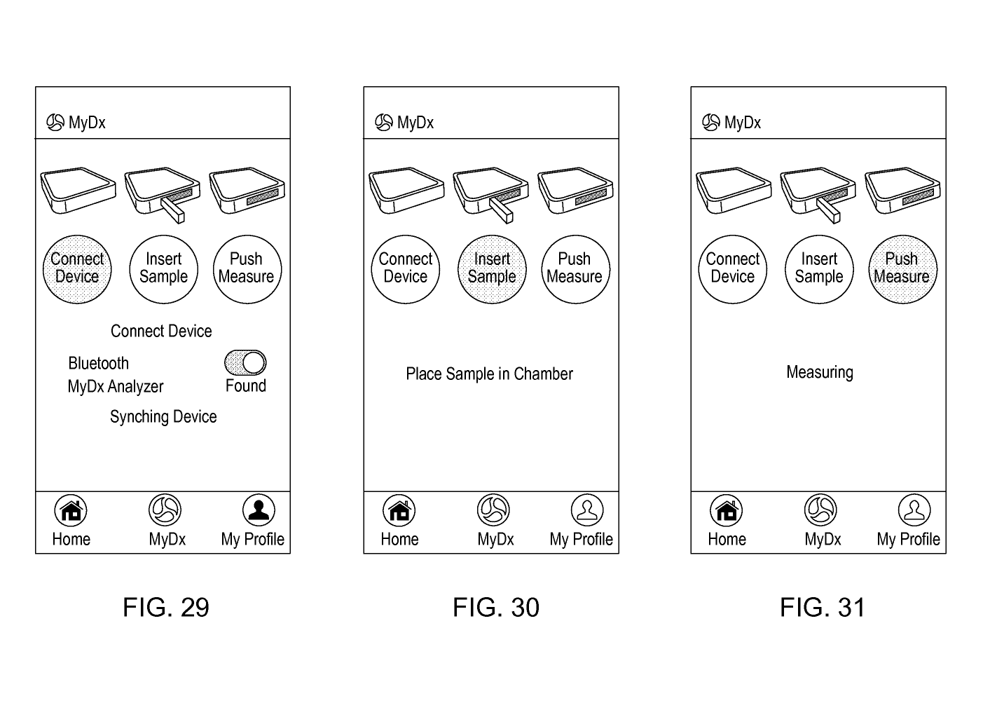

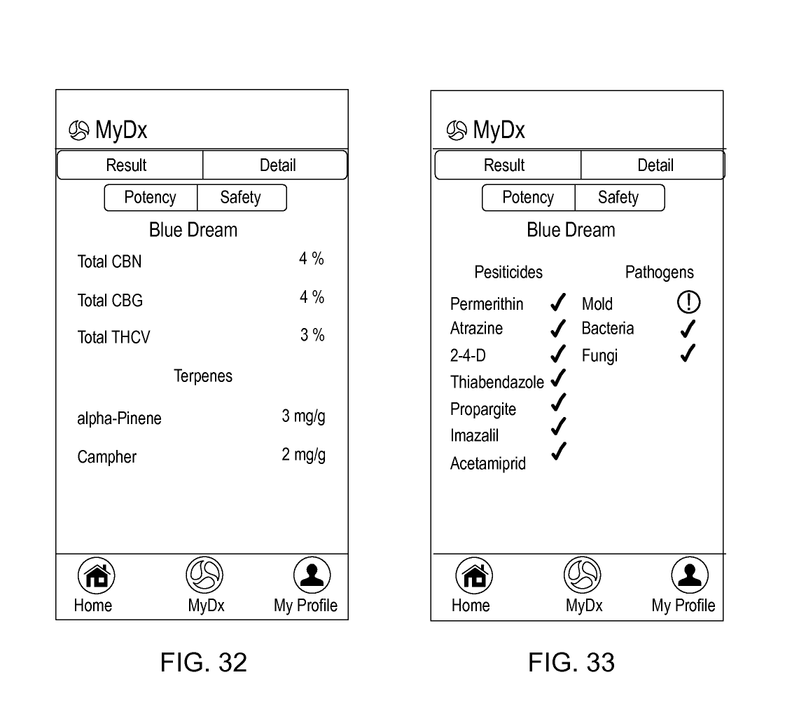

Measurement mode. The user selects the device using a Bluetooth wireless connection (FIG. 29). The user touches an insert sample icon (FIG. 30). The user introduces a sample of the Cannabis product into the sample preparation module and closes the top to the sample preparation module. The user inserts the sample preparation module into the device. The user touches a push measure icon and the device initiates the sample detection process. (FIG. 31) The sample is heated to temperature sufficient to release volatile components to be detected. The analytes are detected by the sensor array and quantified. The information is transmitted to the smartphone. In the potency mode, the smartphone displays the levels of active analytes such as cannabinoids and terpenoids (FIG. 32). In the safety mode, the smartphone alerts the user to impermissible levels of pesticides or pathogens (FIG. 33).

### Example 11

### Alternate User Interfaces for Cannabis Treatment

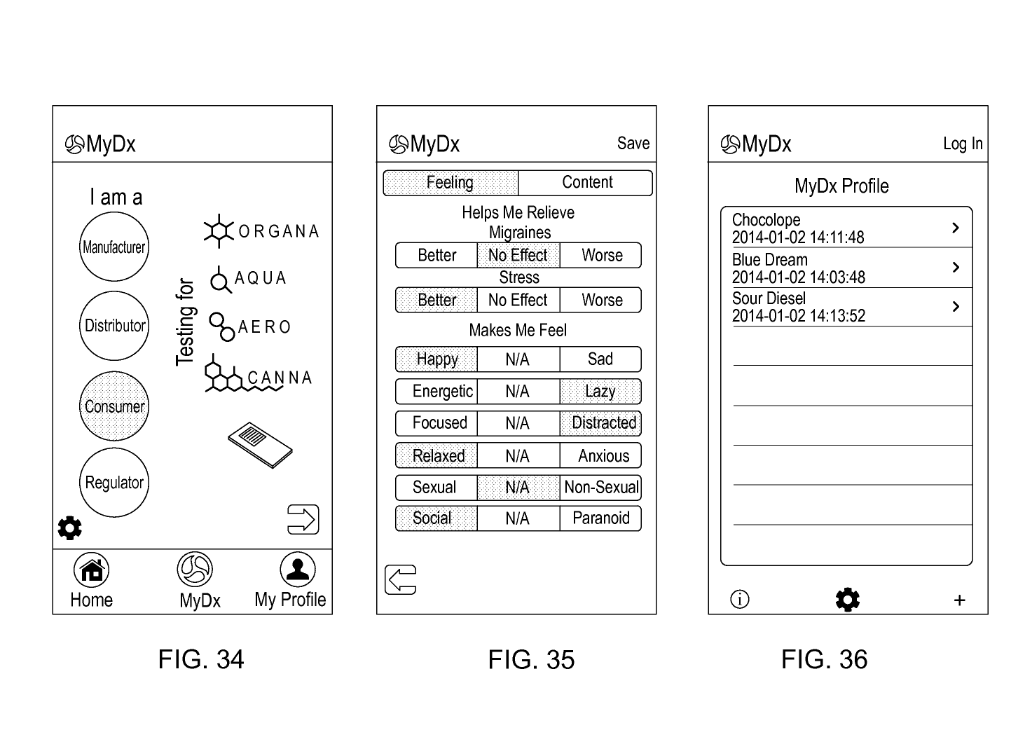

The smartphone interface presents a home screen to select among various user statuses such as manufacturer, distributor, consumer or regulator (FIG. 34). The user touches the icon for consumer. The smartphone interface presents different sample types for testing: organa (organic food sample), aqua (water sample), aero (air sample) and canna (cannabis). The user touches the icon for Cannabis.

The smartphone interface presents a pick list of diseases, disorders and preferred emotional states and selects those that are relevant (FIG. 35). The user touches the desired selections.

The smartphone interface presents a list of prior samples that have been profiled with date and time stamps (FIG. 36).

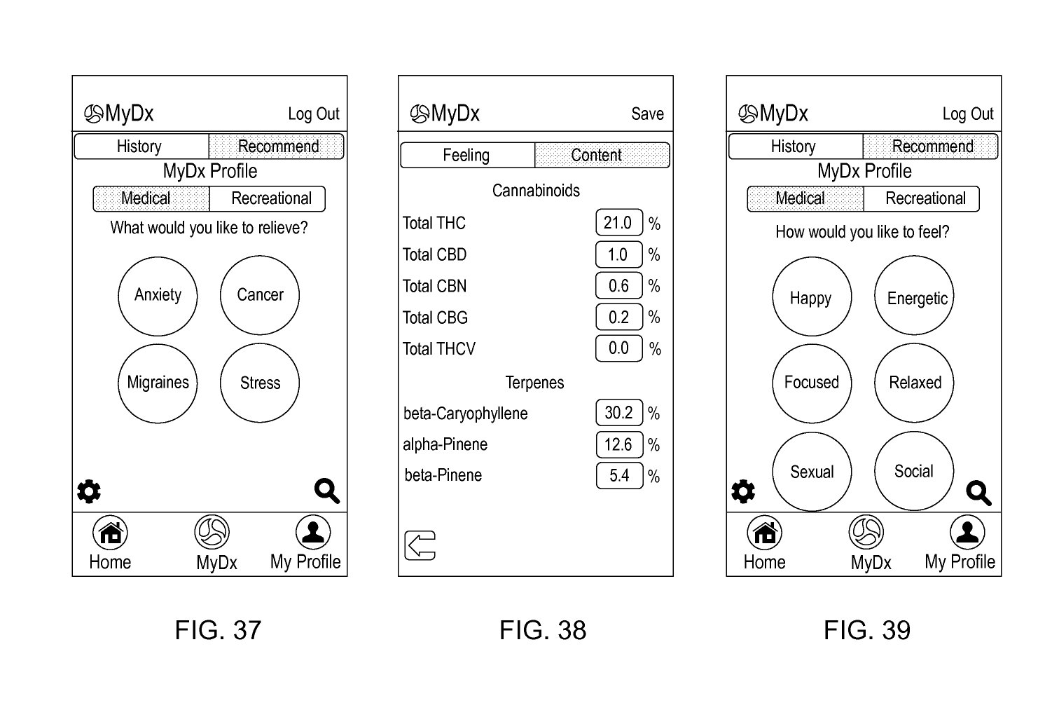

The smartphone interface presents diseases or disorders as alternate selections (FIG. 37). The user touches the icon corresponding to the disease or disorder he or she wishes to relieve.

The smartphone interface presents icons corresponding to a listing of analytes, Cannabanoids and Terpenes, with entry boxes for user input of concentrations of each (FIG. 38). The user inputs the desired concentration for each to generate an analyte profile. The smartphone interface presents a listing of Cannabis strains that possess the analyte profile.

The smartphone interface presents alternative emotional states with the question: “How would you like to feel?” (FIG. 39) The user selects the desired emotional state and the smartphone interface presents a list of Cannabis strains that have been correlated with that emotional state.

All patents and patent applications identified herein are incorporated by reference.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

1. A device for analyte detection comprising a sample preparation module, the sample preparation module comprising a volatilization element, in fluid communication with a detection module, the detection module comprising a sensor array comprising a plurality of sensors for detecting a plurality of analytes, wherein the sensor array is selected from the group consisting of: a conducting polymer sensor array, a carbon nanotube sensor array, a MEMS cantilever sensor array, a surface acoustic wave sensor array, a metal-oxide sensor array, an electrochemical sensor array, a quartz crystal microbalance sensor array, a colorimetric sensor array, a fluorescence sensor array, an infrared sensor array, a MOSFET sensor array and a catalytic bead sensor array.

2. The device according to claim 1 further comprising a distribution module, the distribution module comprising one or more control valves and one or more connection ports wherein the operation of at least one control valve is controlled by information received from the sensor array.

3. The device according to claim 1, wherein the volatilization element is selected from the group consisting of: a heating apparatus, a sonication apparatus, a stirring apparatus, a grinding apparatus and a mechanical shearing apparatus.

4. (canceled)

5. The device according to claim 1, further comprising a mobilization module that directs flow of the volatile component from the sample preparation module to the detection module.

6. The device according to claim 5, wherein the mobilization module comprises a pump.

7. The device according to claim 6, wherein the pump is selected from the group consisting of: a displacement pump, a peristaltic pump, a diaphragm pump and a vacuum driven pump.

8. The device according to claim 1, further comprising a filter.

9. The device according to claim 8, wherein the filter comprises material selected from the group consisting of: carbon, polyester, polycarbonate, cellulose nitrate, mixed cellulose ester, nylon, polytetrafluoroethylene, polypropylene, aluminum oxide, polyether sulfone, and nitrocellulose.

10. The device according to claim 2, wherein the one or more control valves are selected from the group consisting of: a rotary valve, a solenoid valve, a pinch valve, and a hydrogel valve.

11. The device according to claim 2, wherein the one or more connection ports are connected to one or more components selected from the group consisting of: a detection module, a dispensing component module and a sensor device.

12. (canceled)

13. A method of analyte detection comprising (a) volatilizing a sample resulting in a volatile component, (b) separating the volatile component from the remaining sample, and (c) contacting the volatile component with a sensor array comprising a plurality of sensors for detecting a plurality of analytes, wherein the sensor array detects the analyte in the volatile component when the analyte is present in sufficient concentration, wherein the sensor array is selected from the group consisting of: a conducting polymer sensor array, a carbon nanotube sensor array, a MEMS cantilever sensor array, a surface acoustic wave sensor array, a metal-oxide sensor array, an electrochemical sensor array, a quartz crystal microbalance sensor array, a colorimetric sensor array, a fluorescence sensor array, an infrared sensor array, a MOSFET sensor array and a catalytic bead sensor array.

14. The method according to claim 13, wherein the volatilizing of the sample comprises a process selected from the group consisting of: heating, sonicating, stirring, grinding and mechanical shearing.

15. The method according to claim 14, wherein the volatilizing of the sample comprises heating and the temperature is modulated to selectively volatilize at least one analyte.

16. The method according to claim 13, further comprising a filtration step between volatilizing the sample and detecting the analyte in the volatile component.

17. The method according to claim 13, wherein the sample is selected from the group consisting of: an environmental sample, a biological specimen or a pharmaceutical preparation.

18. The method according to claim 17, wherein the sample comprises Cannabis.

19. The method according to claim 18, wherein the analyte is selected from the group consisting of: a cannabinoid, a terpenoid, and a flavonoid.

20. The method according to claim 19, wherein the cannabinoid is selected from the group consisting of: Tetrahydrocannabivarin (THCV), Delta-8-tetrahydrocannabinol (delta-8-THC), Delta-9-tetrahydrocannabinol (THC), Cannabichromene (CBC), Cannabidiol (CBD), Cannabigerol (CBG), and Cannabinol (CBN).

21. The method according to claim 19, wherein the terpenoid is selected from the group consisting of: Linalool, d-Limonene, Myrcene, alpha-Pinene, and Beta-Caryophyllene.

22. The method according to claim 19, wherein the flavanoid is selected from the group consisting of: Beta-sitosterol, Apigenin, Cannflavin A, and Quercetin.

23-54. (canceled)
